(19) United States
(12) Patent Application Publication
(10) Pub. No.: US 2009/0252738 A1
(43) Pub. Date: Oct. 8, 2009

(54) Compositions and methods of therapy for cancers characterized by expression of the tumor-associated antigen mn/ca ix

(75) Inventors: Justin Wong, Oakland, CA (US); Jill Winter, Richmond, CA (US); Guita Lalehzadeh, Oakland, CA (US); Robert Warne, San Leandro, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: Novartis Vaccines and Diagnostics, Inc., Emeryville, CA (US)

(21) Appl. No.: 10/525,535

(22) PCT Filed: Aug. 25, 2003
(86) PCT No.: PCT/US03/26612

§ 371 Date: Mar. 6, 2007

Related U.S. Application Data
(60) Provisional application No. 60/405,577, filed on Aug. 23, 2002.

(51) Int. Cl. A61K 39/395; G01N 33/53; C07K 2/00; A61K 38/00; C07K 14/00; C07K 16/18; A61P 35/00

(52) U.S. Cl. 424/141.1; 435/7.1; 530/300; 514/2; 530/350; 530/388.1

(57) ABSTRACT

Compositions and methods useful in inhibiting proliferation of CA IX+preneoplastic or neoplastic cells in a mammal are provided. The compositions are antagonist anti-CA IX antibodies and other inhibitory agents that target carbonic anhydrase activity of CA IX on these cells. The antagonist anti-CA IX antibodies or antigen-binding fragments thereof are specifically reactive with an inhibitory epitope of CA IX or biologically active variant thereof. Formation of an antibody-antigen complex between the antagonist anti-CA IX antibodies or antigen-binding fragments thereof and the respective inhibitory epitopes results in inhibition of carbonic anhydrase activity of CA IX or biologically active variant thereof. Other small molecule agents that inhibit carbonic anhydrase activity of CA IX or biologically active variant thereof and screening assays for identifying such agents are also provided. The antagonist anti-CA IX antibodies, antigen-binding fragments thereof, and other CA IX inhibitory agents identified herein are useful in the treatment of cancers characterized by the expression of the CA IX tumor-associated antigen.

### FIELD OF THE INVENTION

The present invention is directed to compositions and methods of therapy for the treatment of cell proliferative disorders, more particularly antibodies and small molecules that target MN/CA IX enzymatic activity on neoplastic cells that express this protein and methods of their use in treatment of cancers characterized by expression of this tumor-associated antigen.

### BACKGROUND OF THE INVENTION

Carbonic anhydrases (CAs) catalyze the interconversion of carbon dioxide and bicarbonate. These enzymes play a key role in physiological processes involving water and electrolyte balance, CO₂ and HCO₃⁻ transport, and pH regulation. The novel membrane-associated CA isoenzyme CA IX was initially described as the tumor-associated membrane antigen designated MN. Recognition of MN as being a putative cell-adhesion molecule that has an extracellular domain with the essential structural features and activity of CAs (Pastorek et al. (1994) Oncogene 9:2877-2888; Opavsky et al. (1996) Genomics 33:480-487; Zavada et al. (1997) Intl. J. Oncol. 10:857-863) resulted in the classification of MN as the ninth member of the carbonic anhydrase family, renamed CA IX. Since then, a kidney-cancer-associated antigen designated G250 has been cloned and identified as a transmembrane protein identical to the tumor-associated antigen MN/CA IX (Grabmaier et al. (2000) Int. J. Cancer 85:865-870).

Though originally detected in the human cervical carcinoma cell line HeLa and in a number of human carcinomas, CA IX (MN/G250) is also present in normal gastric, intestinal, and biliary mucosa (Pastorekova et al. (1997) Gastroenterology 112:398-408), more notably in rapidly proliferating normal cells in the small intestine (Saarnio et al. (1998) J. Histochem. Cytochem. 46:497-504). The presence of CA IX protein is almost 100% associated with cervical carcinomas (Liao et al. (1994) Am J. Pathol. 145:598-609), esophagus carcinomas (Turner et al. (1997) Hum. Pathol. 28:740-744), and with renal clear cell carcinomas (Liao et al. (1997) Cancer Res. 57:2827-2831) in human patients. It has also been detected in a high percentage of colorectal (Saarnio et al. (1998) Am. J. Pathol. 153:279-285) and lung (Vermylen et al. (1999) Eur. Respir. J. 14:806-811) carcinomas of human patients.

Expression of CA IX in vitro is upregulated by cell density in HeLa cells and correlates with tumorigenicity in HeLa cell/fibroblast cell hybrids (Zavada et al. (1993) Int. J. Cancer 54:268-274). Further, its expression in NIH3T3 cells promotes cell proliferation (Pastorek et al. (1994) Oncogene 9:2877-2888). Conditions of hypoxia induce expression of CA IX in tumors and cultured tumor cells, indicating that this protein may be a biomarker for hypoxia in some tumors (Ivanov et al. (2001) Am. J Pathol. 158(3):905-919; Beasley et al. (2001) Cancer Res. 61(13):5262-5267). This correlation between CA IX expression, hypoxia, and extracellular acidification have led to the suggestion that expression of CA IX may help to maintain the extracellular acidic pH in tumors, thereby providing a conducive environment for tumor growth and proliferation as well as enhancing tumor resistance to radiotherapy and chemotherapy (Ivanov et al. (2001) Am. J. Pathol. 158(3):905-919; Beasley et al. (2001) Cancer Res. 61(13):5262-5267).

Given its key role in tumor proliferation and progression, compositions and methods for inhibiting the activity of CA IX are needed to provide effective treatment for cancers that are characterized by expression of this protein.

### BRIEF SUMMARY OF THE INVENTION

Compositions and methods useful in inhibiting proliferation of CA IX+preneoplastic or neoplastic cells in a mammal are provided. The compositions are antagonist anti-CA IX antibodies and other inhibitory agents that target carbonic anhydrase activity of CA IX on these cells. The antagonist anti-CA IX antibodies or antigen-binding fragments thereof specifically react with an inhibitory epitope of CA IX or biologically active variant thereof such that an antibody-antigen complex is formed, whereby the formation of this complex results in inhibition of carbonic anhydrase activity of CA IX or biologically active variant thereof. This activity is essential for transformation and proliferation of preneoplastic and neoplastic cells of cancers and other proliferative disorders characterized by expression of this tumor-associated antigen. In one embodiment, the antagonist anti-CA IX antibodies are monoclonal antibodies. Suitable monoclonal antibodies are those having human constant regions, those also having wholly or partially humanized framework regions, and those that are fully human antibodies, or antigen-binding fragments thereof that specifically bind to the inhibitory epitope of interest on CA IX or biologically active variant thereof such that the formation of the antibody-antigen complex results in inhibition of carbonic anhydrase activity of the CA IX protein of interest, for example, human CA IX. Other small molecule agents that inhibit carbonic anhydrase activity of CA IX or biologically active variant thereof and screening assays for identifying such agents are also provided. The antagonist anti-CA IX antibodies, antigen-binding fragments thereof, and other CA IX inhibitory agents identified herein are useful in the treatment of cancers characterized by the expression of the CA IX tumor-associated antigen.

Methods for treating a cancer that is characterized by expression of carbonic anhydrase IX (CA IX) in a mammal are also encompassed. The method comprises administering a therapeutically effective amount of an agent that inhibits carbonic anhydrase activity of CA IX in a mammal in need thereof. Agents of the invention include antagonist anti-CA IX antibodies, and antigen-binding fragments of said antagonist anti-CA IX antibody, peptides, peptoids, and small organic molecules. The anti-CA IX antibodies or antigen binding fragments thereof specifically react with an inhibitory epitope of CA IX to form an antibody-antigen complex, whereby the formation of said complex results in inhibition of carbonic anhydrase activity of said CA IX. The agents of the invention find use in inhibiting proliferation of neoplastic cells that are characterized by expression of CA IX protein. The agents are administered in a therapeutically effective dose to inhibit carbonic anhydrase activity of CA IX.

The invention further comprises methods for screening and identifying agents that inhibit carbonic anhydrase activity of a CA IX polypeptide having a functional carbonic anhydrase domain. The agents are identified by combining an agent to be tested with a cell expressing a CA IX polypeptide under conditions suitable for detecting carbonic anhydrase activity and assessing the ability of an agent to inhibit carbonic anhydrase activity. Inhibition of carbonic anhydrase activity by the agent indicates that the agent is an inhibitor. Agents may also be identified by combining an agent to be tested with a composition comprising an isolated or recombinantly produced CA IX polypeptide under conditions suitable for detecting carbonic anhydrase activity and assessing the ability of the agent to inhibit carbonic anhydrase activity. These agents may be used in compositions or formulated into pharmaceutical compositions for use in the methods of the invention.

### BRIEF DESCRIPTION OF THE DRAWINGS

FIG. 1 shows a fluorescein diacetate assay used to detect carbonic anhydrase activity of CA IX in a cell-free high-throughput screening assay.

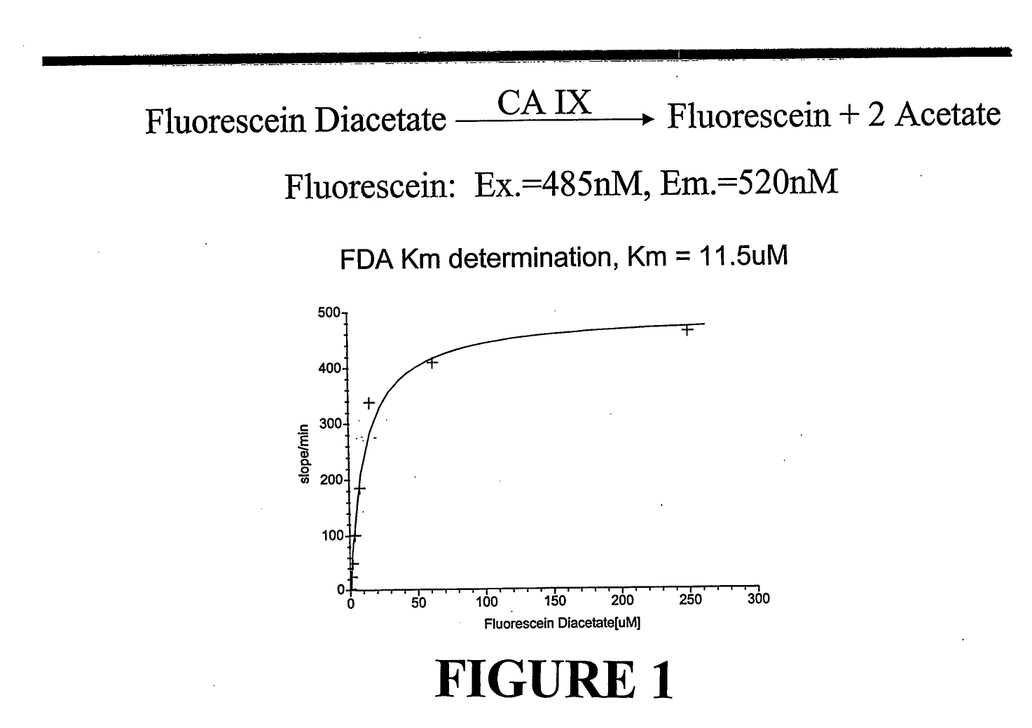

### DETAILED DESCRIPTION OF THE INVENTION

The present invention is directed to compositions and methods for treating a mammal having a cancer characterized by expression of carbonic anhydrase IX (also known as MN and G250; referred to herein as CA IX). Compositions include anti-CA IX antibodies and antigen-binding fragments thereof that specifically react with an epitope of CA IX such that an antibody-antigen complex is formed, whereby the formation of this complex results in inhibition of carbonic anhydrase activity of CA IX. Such epitopes are referred to herein as “inhibitory epitopes.” Inhibitory epitopes can comprise contiguous amino acid residues (i.e., residues within the epitope are arranged sequentially one after another in a linear fashion), discontinuous amino acid residues (referred to herein as “discontinuous epitopes; residues within these epitopes are not arranged sequentially), or both contiguous and discontinuous amino acid residues of the CA IX polypeptide or variant thereof, and the residues making up the epitope can reside within the CA IX carbonic anhydrase domain and/or outside the CA IX carbonic anhydrase domain, so long as the formation of the antibody-antigen complex with the anti-CA IX antibody or antigen-binding fragment thereof results in reduction or inhibition of the carbonic anhydrase activity of CA IX. In some embodiments, the inhibitory epitopes are epitopes comprising contiguous amino acid residues of the carbonic anhydrase domain of CA IX, epitopes comprising discontinuous amino acid residues of the carbonic anhydrase domain of CA IX, and epitopes comprising both contiguous and discontinuous residues of the carbonic anhydrase domain. Binding of an anti-CA IX antibody or antigen-binding fragment thereof to inhibitory epitopes of CA IX as described herein may prevent CA IX from assuming an appropriate structural form or conformation necessary for mediating carbonic anhydrase activity, may physically inhibit CA IX from complexing with inorganic co-factors necessary for carbonic anhydrase activity, or may physically inhibit CA IX from complexing with the appropriate enzymatic substrate, though other mechanisms of inhibition are also encompassed. For purposes of the present invention, the anti-CA IX antibodies of the invention are hereinafter referred to as “antagonist anti-CA IX antibodies.” These antagonist anti-CA IX antibodies and other inhibitory agents as noted elsewhere herein are useful in treating a subject having a cancer that is characterized by expression of the CA IX tumor-associated antigen.

CA IX, also known as the MN and G250 tumor-associated antigens, is an N-glycosylated transmembrane protein encoded by the MN gene. The coding sequence for human CA IX (see SEQ ID NO:1) and the translated amino acid sequence for this protein (SEQ ID NO:2) are known in the art. See, for example, U.S. Pat. Nos. 6,204,370 and 5,989,838, herein incorporated by reference. Human CA IX comprises a signal peptide, a proteoglycan-like domain, a carbonic anhydrase domain, a transmembrane domain, and an intracellular (also referred to as intracytoplasmic) domain at the C-terminus. With the exception of the starting position of the signal peptide (i.e., residue 1), the residue positions denoting the location of these domains within the CA IX protein vary within the reported literature, generally by ±1-3 residues at either end of any given domain. For purposes of the present invention, this variation is accounted for with the use of the term “about.” In this manner, the term “about” with respect to the residue denoting the beginning or end of each of these domains encompasses 1-3 residues on either side of the designated position. Thus, for example, where a domain is designated a location of about residues 38-134 of the human CA IX amino acid sequence, it is intended that the domain encompasses a starting location at residue 38±1-3 residues and encompasses an ending location at residue 134±1-3 residues. In this example, the starting location would encompass residue 35, 36, 37, 38, 39, 40, or 41 of the human sequence, and the ending location would encompass residue 131, 132, 133, 134, 135, 136, or 137 of the human sequence. For purposes of the present invention, the residue locations of the various domains of the human CA IX protein are defined in accordance with this foregoing description.

The human CA IX protein consists of a signal peptide (corresponding to residue 1 to about residue 37 of SEQ ID NO:2), a proteoglycan-like domain (corresponding to about residues 38-134 of SEQ ID NO:2), a carbonic anhydrase domain (corresponding to about residues 135-414 of SEQ ID NO:2), a transmembrane domain (corresponding to about residues 415-433 of SEQ ID NO:2), and an intracellular C-terminus (corresponding to about residues 434-459 of SEQ ID NO:2). The functional region of the proteoglycan-like domain resides at about residues 53-111, while the functional region of the carbonic anhydrase domain resides at about residues 141-389. Three zinc-liganded histidine residues obligatory for carbonic anhydrase activity are located within the carbonic anhydrase (CA) domain (at residues 226, 228, and 251 of SEQ ID NO:2). Antigenic region analysis of the full-length human CA IX protein predicts an antigenic region within the carbonic anhydrase domain, with the region of antigenicity residing at about residues 229-256 of SEQ ID NO:2.

The CA IX tumor-associated antigen was initially identified as being expressed in some human tumor cell lines in vitro, for example, by T24 (bladder carcinoma), HeLa (cervical carcinoma), SK-Mel 1477 (melanoma), and T47D (mammary carcinoma). CA IX is also produced by cells of some human cancers in vivo, for example, by cells of ovarian and endometrial carcinomas, uterine cervical carcinomas, renal cell carcinomas (RCC), colorectal cancer (CRC), and lung cancer, as well as cells of some benign neoplasias such as mammary papillomas. CA IX is not found in non-tumorigenic hybrid cells, and is generally not found in the cells of normal tissues. An exception resides in its expression within normal gastric, intestinal, and biliary mucosa. The CA IX gene is strongly correlated with tumorigenesis and may be a causative agent.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth, including tumors and neoplastic growth (i.e., neoplasms). Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectalcancer, endometrial carcinoma, salivary gland carcinoma, kidney cancer, liver cancer, vulval cancer, thyroid cancer, hepatic carcinoma, and various types of head and neck cancer.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. The “pathology” of cancer includes all phenomena that compromise the well being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

The CA IX protein and gene are described in e.g., U.S. Pat. Nos. 6,204,370 and 5,989,838. Although evidence to date pointed to CA IX as having a role in transforming cells, it has been unknown prior to the present invention which functional domain of this protein is responsible for its role in cell transformation and tumorigenesis. The present invention is based upon the discovery that this role resides within the carbonic anhydrase domain. Thus, as shown in the examples disclosed herein, cells expressing a functional carbonic anhydrase domain of the CA IX protein develop a transformed phenotype. By “functional” carbonic anhydrase domain is intended that the domain is expressed and has functional carbonic anhydrase activity. Development of a transformed phenotype is exemplified by one or more activities selected from the group consisting of promotion of cell proliferation, faster doubling times, enhanced DNA synthesis, induced spindle-shaped morphology, increased refractility, decreased adherence, lost capacity for growth arrest, chaotic growth pattern with higher saturation densities, decreased growth factor dependence, growth in soft agar using the soft agar assay described herein (a measure of anchorage independence), increased expression of CA IX as cell density increases, and ability to grow in nude mice (i.e., tumorigenicity).

Compositions and methods of the invention are directed to the inhibition of carbonic anhydrase activity of CA IX protein or biologically active variant thereof on preneoplastic or neoplastic cells as described herein below. When administered to a subject in need of treatment, compositions of the invention effectively reduce or inhibit carbonic anhydrase activity of CA IX or biologically active variant thereof by 5%, 10%, 15%, 20%, 25%, 30%, 35%, preferably 40%, 45%, 50%, 55%, 60%, more preferably 70%, 75%, 80%, 85%, and most preferably 90%, 95%, 99%, or 100%. Methods for measuring the ability of a candidate antibody or candidate test agent to inhibit carbonic anhydrase activity of a CA IX protein are known in the art. See, for example, the assays described herein below, though any assay available in the art for measuring CO₂ release or conversion in the presence of the candidate inhibitory agent can be used to detect the ability of the candidate agent to inhibit CA IX carbonic anhydrase activity. Reduction or inhibition of this activity on preneoplastic cells prevents their phenotypic transformation into tumorogenic cells. Reduction or inhibition of this activity on neoplastic cells can inhibit their continued proliferation or result in overall reduction in tumor size and tumor burden.

Thus, compositions of the invention can be used to inhibit carbonic anhydrase activity of CA IX at the cell surface of preneoplastic or neoplastic cells expressing this cell-surface antigen, thereby limiting carbonic anhydrase activity in the extracellular environment. Where the composition is an antagonist antibody of the invention, such inhibition occurs by the formation of an antibody-antigen complex as described elsewhere herein. Depending upon the location of the epitope within the CA IX protein, formation of the antibody-antigen complex between the epitope and an antagonist anti-CA IX antibody of the invention can result in a complex that resides extracellularly. Alternatively, formation of the antibody-antigen complex can result in internalization of the CA IX cell-surface protein, thereby sequestering the CA IX molecule inside the cell. Where internalization occurs, the antagonist anti-CA IX antibody can also be used as a delivery mechanism to deliver antibody-conjugated therapeutic moieties to the inside of CA IX-bearing tumor cells. Suitable therapeutic moieties that can be conjugated to the antagonist anti-CA IX antibodies of the invention for subsequent delivery to the inside of CA IX-bearing tumor cells are described herein below and include, but are not limited to, cytotoxins, chemotherapeutics, radio-metal ions, and the like.

Any agent that inhibits carbonic anhydrase activity of CA IX or biologically active variant thereof on CA IX+preneoplastic or neoplastic cells can be used in the methods of the present invention to prevent phenotypic transformation into tumorogenic cells and/or to inhibit proliferation of these cells. These agents find use in the treatment of subjects having a cancer characterized by expression of this tumor-associated antigen. Suitable inhibitory agents can be identified using a variety of screening assays, including these disclosed herein. In one embodiment, suitable agents are identified by monitoring their ability to inhibit cells expressing the CA IX molecule of interest from displaying the transformed phenotype using the soft agar assay described herein below. Suitable inhibitory agents include the antagonist anti-CA IX antibodies disclosed herein, and peptides, peptoids, and small organic molecules that target activity of the carbonic anhydrase (CA) domain of CA IX.

### Antagonist Anti-CA IX Antibodies

Suitable anti-CA IX antibodies of the invention are referred to herein as “antagonist” anti-CA IX antibodies in view of their ability to inhibit carbonic anhydrase activity of a CA IX polypeptide as noted above. In this manner, the antagonist anti-CA IX antibodies of the invention and antigen-binding fragments thereof are specifically reactive with an inhibitory epitope of CA IX such that an antibody-antigen complex is formed, whereby the formation of this complex results in inhibition of carbonic anhydrase activity of CA IX. As noted above, inhibitory epitopes can comprise contiguous amino acid residues, discontinuous amino acid residues, or both contiguous and discontinuous amino acid residues of the CA IX polypeptide or variant thereof, and the residues making up the epitope can reside within the CA IX carbonic anhydrase domain and/or outside the CA IX carbonic anhydrase domain, so long as the formation of the antibody-antigen complex with the anti-CA IX antibody or antigen-binding fragment thereof results in reduction or inhibition of the carbonic anhydrase activity of CA IX. In some embodiments, the inhibitory epitopes are epitopes comprising contiguous amino acid residues of the carbonic anhydrase domain of CA IX, epitopes comprising discontinuous amino acid residues of the carbonic anhydrase domain of CA IX, and epitopes comprising both contiguous and discontinuous residues of the carbonic anhydrase domain.

By “specifically reactive” or “specifically reacts” is intended that the antibody that recognizes a particular region of the CA IX protein that comprises the inhibitory epitope of interest forms a specific antibody-antigen complex with that portion of the CA IX protein corresponding to the inhibitory epitope of interest when the antibody comes into contact with that protein, either in an in vitro or in vivo setting. Methods for detecting the formation of such antibody-antigen complexes are well known in the art. For example, candidate antagonist anti-CA IX antibodies that have been prepared against a CA IX protein or biologically active portion thereof that comprises the CA domain of the CA IX protein of interest can be tested for formation of an antibody-antigen complex using a number of well-defined diagnostic assays, such as conventional immunoassay formats to detect and/or quantitate antigen-specific antibodies. Such assays include, for example, enzyme immunoassays, e.g., enzyme-linked immunosorbant assays (ELISA), cell-based assays, flow cytometry, radioimmunoassays, and immunohistochemical staining. Numerous competitive and non-competitive protein binding assays are known in the art and many are commercially available. A representative assay to detect anti-CA IX antibodies specific to the inhibitory epitopes identified herein, for example inhibitory epitopes comprising residues within the carbonic anhydrase domain, is a competition assay in which labeled CA IX polypeptide comprising residues within the CA domain is precipitated by candidate antibodies in a sample, for example, in combination with monoclonal antibodies raised against one or more epitopes comprising residues within this domain. Anti-CA IX antibodies that specifically react with an epitope of interest, i.e., an inhibitory epitope of CA IX, can be identified by screening a series of antibodies prepared against a CA IX protein or fragment of the protein comprising the particular epitope of the CA IX protein of interest. For example, for human CA IX, epitopes of interest include inhibitory epitopes comprising contiguous and/or discontinuous amino acid residues of human CA IX of SEQ ID NO:2. Such inhibitory epitopes of human CA IX include, for example, those inhibitory epitopes comprising contiguous and/or discontinuous amino acid residues residing within the CA domain corresponding to about residues 135-414 of SEQ ID NO:2. Alternatively, competition assays with previously identified suitable antagonist anti-CA IX antibodies could be used to select monoclonal antibodies comparable to the previously identified antibodies.

Antibodies employed in such immunoassays may be labeled or unlabeled. Unlabeled antibodies may be employed in agglutination; labeled antibodies may be employed in a wide variety of assays, employing a wide variety of labels. Detection of the formation of an antibody-antigen complex between an anti-CA IX antibody and an epitope of interest can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like. See for example, U.S. Pat. Nos. 3,766,162; 3,791,932; 3,817,837; and 4,233,402.

Where the CA IX protein is human CA IX, the antagonist anti-CA IX antibodies of the invention are specifically reactive with an inhibitory epitope comprising contiguous and/or discontinuous residues of the human CA IX protein, and can comprise residues within the carbonic anhydrase (CA) domain of human CA IX (i.e., about residues 135-414 of SEQ ID NO:2), residues outside the CA domain, or residues residing inside and outside the CA domain of human CA IX. Such inhibitory epitopes can correspond to a length of at least 5 residues, at least 8 residues, at least 10, 15, 20, 25, 30, or at least 35 residues of the human CA IX protein.

In some embodiments, the antagonist anti-CA IX antibodies are specifically reactive with an inhibitory epitope comprising contiguous and/or discontinuous residues of the carbonic anhydrase (CA) domain of human CA IX, which is located at about residues 135-414 of SEQ ID NO:2 and encompasses the functional region of this domain, i.e., about residues 141-389 of SEQ ID NO:2. In one embodiment, the antagonist anti-CA IX antibodies are specifically reactive with an inhibitory epitope corresponding to at least 5 residues, at least 8 residues, at least 10, 15, 20, 25, 30, or at least 35 residues of the CA domain of human CA IX protein, i.e., about residues 135-414 of SEQ ID NO:2. In these embodiments, the residues making up the inhibitory epitope can all be contiguous, can all be discontinuous, or can be a mixture of contiguous and discontinuous residues. Examples of such inhibitory epitopes are those inhibitory epitopes corresponding to a group of contiguous and/or discontinuous amino acid residues selected from residues 200 to 300, residues 210 to 260, residues 220 to 255, and residues 229 to 256 of SEQ ID NO:2. In one embodiment, the inhibitory epitope has a length of about 5, 8, 10, 15, 20, 25, 30, or 35 residues, and comprises at least one of the three essential zinc-liganded histidine residues of the CA domain, i.e., at least one of residues 226, 228, and 251 of SEQ ID NO:2. In other embodiments, the inhibitory epitope has a length of about 5, 8, 10, 15, 20, 25, 30, or 35 residues, and comprises at least one of residues 229 to 256 of SEQ ID NO:2. In such embodiments, the inhibitory epitope can comprise, for example, 1, 3, 5, 8, 10, 12, 15, 18, 20, 22, 25, or even 28 of the residues within residues 229 to 256 of SEQ ID NO:2. Where the inhibitory epitope comprises more than one of, but less than 28 of, residues 229 to 256 of SEQ ID NO:2, these residues can all be contiguous, can all be discontinuous, or can be a mixture of contiguous and discontinuous residues.

Where the CA IX protein is a biologically active variant of human CA IX as defined herein below, the antagonist anti-CA IX antibodies of the invention are specifically reactive with an inhibitory epitope comprising contiguous and/or discontinuous residues of the CA IX polypeptide variant. Such an inhibitory epitope can comprise residues within the functional CA domain of the CA IX polypeptide variant (i.e., the functional CA domain that is homologous to the functional domain of human CA IX residing at about residues 135-414 of SEQ ID NO:2 when the amino acid sequence for the CA domain of the CA IX polypeptide variant is optimally aligned against the amino acid sequence for the CA domain of human CA IX using sequence alignment methods noted herein below), residues outside the functional CA domain, or residues residing inside and outside the functional CA domain of the CA IX polypeptide variant. Such inhibitory epitopes can correspond to a length of at least 5 residues, at least 8 residues, at least 10, 15, 20, 25, 30, or at least 35 residues of the CA IX polypeptide variant.

In some embodiments, the antagonist anti-CA IX antibodies of the invention are specifically reactive with an inhibitory epitope of the CA IX polypeptide variant that comprises contiguous and/or discontinuous residues of the functional carbonic anhydrase (CA) domain that is homologous to the functional carbonic CA domain in human CA IX (i.e., about residues 135-414 of SEQ ID NO:2) when the amino acid sequence for the CA domain of the CA IX polypeptide variant is optimally aligned against the amino acid sequence for the CA domain of human CA IX using sequence alignment methods noted herein below. As with human CA IX, the antagonist anti-CA IX antibodies are specifically reactive with an inhibitory epitope corresponding to at least 5 residues, at least 8 residues, at least 10, 15, 20, 25, 30, or at least 35 residues of the functional CA domain of the CA IX polypeptide variant. In these embodiments, the residues making up the inhibitory epitope can all be contiguous, can all be discontinuous, or can be a mixture of contiguous and discontinuous residues. Examples of such inhibitory epitopes are those inhibitory epitopes corresponding to a group of contiguous and/or discontinuous amino acid residues selected from the region of the functional CA domain that is homologous to residues 200 to 300, residues 210 to 260, residues 220 to 255, and residues 229 to 256 of SEQ ID NO:2 when the amino acid sequence for the CA domain of the CA IX polypeptide variant is optimally aligned against the amino acid sequence for the CA domain of human CA IX using sequence alignment methods noted herein below. In one embodiment, the inhibitory epitope of the CA IX polypeptide variant has a length of about 5, 8, 10, 15, 20, 25, 30, or 35 residues, and comprises at least one of the three essential zinc-liganded histidine residues of the functional CA domain, i.e., at least one of the residues homologous to residues 226, 228, and 251 of SEQ ID NO:2 when the amino acid sequence for the functional CA domain of the CA IX polypeptide variant is optimally aligned against the amino acid sequence for the CA domain of human CA IX using sequence alignment methods noted herein below. In other embodiments, the inhibitory epitope of the CA IX polypeptide variant has a length of about 5, 8, 10, 15, 20, 25, 30, or 35 residues, and comprises at least one of the residues of the region of the functional CA domain that is homologous to residues 229 to 256 of SEQ ID NO:2 when the amino acid sequence for the functional CA domain of the CA IX polypeptide variant is optimally aligned against the amino acid sequence for the CA domain of human CA IX using sequence alignment methods noted herein below. In such embodiments, the inhibitory epitope can comprise, for example, 1, 3, 5, 8, 10, 12, 15, 18, 20, 22, 25, or even 28 of the residues within the region of the functional CA domain of the CA IX polypeptide variant that is homologous to residues 229 to 256 of SEQ ID NO:2. Where the inhibitory epitope of the CA IX polypeptide variant comprises more than one of, but less than 28 of, the residues in the region of the functional CA domain that is homologous to residues 229 to 256 of SEQ ID NO:2, these residues can all be contiguous, can all be discontinuous, or can be a mixture of contiguous and discontinuous residues.

Any antagonist anti-CA IX antibody or antigen-binding fragment thereof having the binding characteristics and specificity noted herein is suitable for use in the methods of the present invention. Thus, suitable antagonist anti-CA IX antibodies are specifically reactive with an epitope of CA IX such that an antibody-antigen complex is formed, whereby formation of the complex results in inhibition of CA IX carbonic anhydrase activity by 5%, 10%, 15%, 20%, 25%, 30%, 35%, preferably 40%, 45%, 50%, 55%, 60%, more preferably 70%, 75%, 80%, 85%, and most preferably 90%, 95%, 99%, or 100%. Antagonist anti-CA IX antibodies that are specifically reactive with an inhibitory epitope of interest of human CA IX protein or variant thereof can be prepared against human CA IX protein or the variant CA IX polypeptide, for example, antigenic peptides comprising all or a portion of the carbonic anhydrase domain of a CA IX protein of interest, and identified using immunoassays for detecting antibody-antigen complexes and epitope mapping techniques known in the art. Such immunoassays and epitope mapping techniques include those described in U.S. Pat. Nos. 4,708,871 and 5,635,182, herein incorporated by reference in their entirety, and noted elsewhere herein.

As used herein, “antagonist anti-CA IX antibody” thus encompasses any antibody that specifically recognizes and binds to an epitope of the CA IX tumor-associated antigen whereby formation of the antibody-antigen complex results in inhibition of carbonic anhydrase activity of CA IX. As noted above, epitopes that are recognized by the antagonist anti-CA IX antibodies of the invention are referred to herein as “inhibitory epitopes,” as formation of an antibody-antigen complex between the antagonist anti-CA IX antibody and the inhibitory epitope results in inhibition of carbonic anhydrase activity of CA IX. The antagonist anti-CA IX antibodies of the invention include polyclonal antibodies, monoclonal antibodies, single-chain antibodies, and fragments thereof such as Fab, F(ab′)₂, Fᵥ, and other fragments that retain the antigen binding function of the parent antagonist anti-CA IX antibody, i.e., binding to an inhibitory epitope of CA IX, whereby carbonic anhydrase activity of this protein is inhibited. Polyclonal sera may be prepared by conventional methods. In general, a solution containing the CA IX antigen is first used to immunize a suitable animal, preferably a mouse, rat, rabbit, or goat. Rabbits or goats are preferred for the preparation of polyclonal sera due to the volume of serum obtainable, and the availability of labeled anti-rabbit and anti-goat antibodies. Polyclonal sera can be prepared in a transgenic animal, preferably a mouse bearing human immunoglobulin loci. In one embodiment, Sf9 cells or Tn5 cells expressing CA IX are used as the immunogen. Immunization can also be performed by mixing or emulsifying the antigen-containing solution, preferably in an adjuvant such as Freund's complete adjuvant, and injecting the mixture or emulsion parenterally (generally subcutaneously, intraperitoneally, or intramuscularly). A dose of about 10-200, up to about 500 μg/injection is typically sufficient. Immunization is generally boosted 2-6 weeks later with one or more injections of the protein, preferably emulsified with Freund's incomplete adjuvant. One may alternatively generate antibodies by in vitro immunization using methods known in the art, which for the purposes of this invention is considered equivalent to in vivo immunization. Polyclonal antisera are obtained by bleeding the immunized animal into a glass or plastic container, incubating the blood at 25° C. for one hour, followed by incubating at 4° C. for 2-18 hours. The serum is recovered by centrifugation (e.g., 1,000×g for 10 minutes). About 20-50 ml per bleed may be obtained from rabbits.

Preferably the antibody is monoclonal in nature. By “monoclonal antibody” is intended an antibody obtained from a population of substantially homogeneous antibodies, i.e., the individual antibodies comprising the population are identical except for possible naturally occurring mutations or modifications that may be present in minor amounts. Monoclonal antibodies are highly specific, being directed against a single antigenic site, i.e., for purposes of the present invention, an epitope of CA IX described herein that when bound to an antagonist antibody of the invention forms an antibody-antigen complex, whereby carbonic anhydrase activity of CA IX is inhibited. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from yeast display (Hudson and Souriau (2001) Expert. Opin. Biol. Ther. 1(5):845-855), ribosomal display (Hudson and Souriau (2001) Expert. Opin. Biol. Ther. 1(5):845-855), or phage antibody libraries using the techniques described in, for example, Clackson et al. (1991) Nature 352:624-628; Marks et al. (1991) J. Mol. Biol. 222:581-597, and U.S. Pat. No. 5,514,548; herein incorporated by reference.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution, preferably in an adjuvant such as Freund's complete adjuvant or Freund's incomplete adjuvant, and injecting the mixture or emulsion parenterally. Any method of immunization known in the art may be used to obtain the monoclonal antibodies of the invention. After immunization of the animal, the spleen (and/or optionally, several large lymph nodes) is removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas, antibody can be produced in a cell line such as a CHO cell line or myeloma cell line, as disclosed in U.S. Pat. Nos. 5,545,403; 5,545,405; 5,998,144; and 5,807,715; incorporated herein by reference. Examples of myeloma cell lines include, but are not limited to, Sp2 and NS0 cell lines, and cell lines derived therefrom. Briefly the cell line is transfected with vectors capable of expressing a light chain and a heavy chain, respectively. By transfecting the separate vectors encoding the two proteins, chimeric antibodies can be produced. Another advantage is the correct glycosylation of the antibody. Alternatively, the antibody can be produced by transfecting the cell line with a bicistronic single plasmid encoding the light and heavy chains.

Additionally, the term “anti-CA IX antibody” as used herein encompasses chimeric anti-CA IX antibodies. By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region or portion thereof of the chimeric antibody is most preferably derived from a non-human source and has the desired antigenic specificity to the region of the CA IX protein of interest, for example, human CA IX or biologically active variant thereof, that comprises an inhibitory epitope described therein. The non-human source can be any vertebrate source that can be used to generate antibodies to a human CA IX or material comprising a human CA IX, or more particularly an antigenic peptide of human CA IX comprising an inhibitory epitope described herein, for example, an inhibitory epitope residing within the CA domain. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). As used herein, the phrase “immunologically active” when used in reference to chimeric anti-CA IX antibodies means a chimeric antibody that binds human CA IX, more specifically binds to an inhibitory epitope described herein, for example, an inhibitory epitope comprising contiguous and/or discontinuous residues residing within the CA domain of human CA IX or biologically active variant thereof.

Humanized anti-CA IX antibodies are also encompassed by the term anti-CA IX antibody as used herein. By “humanized” is intended forms of anti-CA IX antibodies that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205; herein incorporated by reference. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596; herein incorporated by reference. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Also encompassed by the term anti-CA IX antibodies are xenogeneic or modified anti-CA IX antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. Nos. 5,877,397 and 5,939,598, herein incorporated by reference.

Fragments of the antagonist anti-CA IX antibodies are suitable for use in the methods of the invention so long as they retain the desired affinity and activity of the full-length antibody. Thus, a fragment of an anti-CA IX antibody will (1) retain the ability to bind to an inhibitory epitope of CA IX as defined elsewhere herein, for example, an inhibitory epitope comprising contiguous and/or discontinuous residues of the CA domain of the CA IX protein of interest, for example, human CA IX or biologically active variant thereof; and (2) exhibit antagonist activity when bound to such an inhibitory epitope of CA IX, thereby inhibiting carbonic anhydrase activity of the CA IX protein. Such fragments are referred to herein as “antigen-binding” fragments.

Suitable antigen-binding fragments of an antibody comprise a portion of a full-length antibody, generally the antigen-binding or variable region thereof. Examples of antibody fragments include, but are not limited to, Fab, F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456; herein incorporated by reference. Generally, the Fv polypeptide further comprises a polypeptide linker between the V_{H} and V_{L} domains that enables the sFv to form the desired structure for antigen binding. For a review of sFv, see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody yeast libraries, ribosomal expression libraries, or phage libraries generated using the techniques described in, for example, McCafferty et al. (1990) Nature 348:552-554 (1990) and U.S. Pat. No. 5,514,548. Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody yeast, ribosomal, or phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

The invention also encompasses de-immunized antagonist anti-CA IX antibodies, which can be produced as described in, for example, International Publication Nos. WO 98/52976 and WO 0034317; herein incorporated by reference. In this manner, residues within the antagonist anti-CA IX antibodies of the invention are modified so as to render the antibodies non- or less immunogenic to humans while retaining their antagonist activity toward carbonic anhydrase activity of CA IX, wherein such activity is measured by assays noted elsewhere herein. Also included within the scope of the invention are fusion proteins comprising an antagonist anti-CA IX antibody of the invention, or a fragment thereof, which fusion proteins can be synthesized or expressed from corresponding polynucleotide vectors, as is known in the art. Such fusion proteins are described with reference to conjugation of antibodies as noted below.

Antagonist anti-CA IX antibodies identified as having the binding characteristics and specificity described herein for use in the methods of the present invention can have sequence variations produced using methods described in, for example, Patent Publication Nos. EP 0 983 303 A1, WO 00/34317, and WO 98/52976, incorporated herein by reference. For example, it has been shown that sequences within the CDR can cause an antibody to bind to MHC Class II and trigger an unwanted helper T cell response. A conservative substitution can allow the antibody to retain binding activity yet lose its ability to trigger an unwanted T cell response. Any such conservative or non-conservative substitutions can be made using art-recognized methods, such as those noted elsewhere herein, and the resulting antibodies will fall within the scope of the invention. The variant antibodies can be routinely tested for antagonist activity, affinity, and specificity using methods described herein.

The antibodies of this invention can also have ADCC and CDC activity in addition to neutralizing activity. Antibodies that have ADCC activity interact with “human effector cells” such as leukocytes that express one or more FcRs and perform effector functions. Preferably, the cells express at least FcyRIII and carry out antigen-dependent cell-mediated cyotoxicity (ADCC) effector function. Examples of human leukocytes that mediate ADCC include peripheral blood mononuclear cells (PBMC), natural killer (NK) cells, monocytes, cytotoxic T cells, and neutrophils; with PBMCs and NK cells being preferred. Antibodies that have ADCC activity are typically of the IgG1 or IgG3 isotype. Note that in addition to isolating IgG1 and IgG3 antibodies, such ADCC-mediating antibodies can be made by engineering a variable region from a non-ADCC antibody or variable region fragment onto an IgG1 or IgG3 isotype constant region.

The terms “Fc receptor” or “FcR” are used to describe a receptor that binds to the Fc region of an antibody. The preferred FcR is a native sequence human FcR. Moreover, a preferred FcR is one that binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain (see Daeron (1997) Annu. Rev. Immunol. 15:203-234). FcRs are reviewed in Ravetch and Kinet (1991) Annu. Rev. Immunol 9:457-92; Capel et al. (1994) Immunomethods 4:25-34; and de Haas et al. (1995) J. Lab. Clin. Med. 126:330-341. Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587; and Kim et al. (1994) J. Immunol. 24:249).

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al. (1996) J. Immunol. Methods 202:163, maybe performed.

Any of the previously described antagonist anti-CA IX antibodies or antibody fragments thereof may be conjugated prior to use in the methods of the present invention. Methods for producing conjugated antibodies are known in the art. Thus, the antagonist anti-CA IX antibody may be labeled using “indirect labeling” or an “indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18:589-603, herein incorporated by reference. Alternatively, the antagonist anti-CA IX antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred. See also, for example, International Publication Nos. WO 00/52031 and WO 00/52473, where a linker is used to attach a radioactive label to antibodies; and the labeled forms of anti-CA IX antibodies described in U.S. Pat. No. 6,015,542; herein incorporated by reference.

Further, an antagonist anti-CA IX antibody (or antigen-binding fragment thereof) may be conjugated to a therapeutic moiety such as a cytotoxin, a therapeutic agent, or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-alpha, interferon-beta, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6), granulocyte macrophase colony stimulating factor (“GM-CSF”), granulocyte colony stimulating factor (“G-CSF”), or other growth factors.

Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; Hellstrom et al. eds. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. (Editrice Kurtis, Milano, Italy, 1985), pp. 475-506; “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp.303-316; and Thorpe et al. (1982) Immunol. Rev. 62:119-158.

Alternatively, an antibody can be conjugated to a second antibody to form an antibody heteroconjugate as described by Segal in U.S. Pat. No. 4,676,980. In addition, linkers may be used between the labels and the antibodies of the invention (see U.S. Pat. No. 4,831,175). Antibodies or, antigen-binding fragments thereof may be directly labeled with radioactive iodine, indium, yttrium, or other radioactive particle known in the art (U.S. Pat. No. 5,595,721). Treatment may consist of a combination of treatment with conjugated and nonconjugated antibodies administered simultaneously or subsequently (WO 00/52031 and WO 00/52473).

The anti-CA IX antibodies of the present invention can bind to amino-acid-residues-specific-epitopes, carbohydrate-specific epitopes, or epitopes formed by both amino acid residues and carbohydrate portions of the molecule, as expressed by the target-bearing tumor cells. If the anti-CA IX antibody carries a cytotoxic or cytostatic agent, it might be preferable that the antibody binds to an epitope that internalizes the antibody-target receptor complex. If the anti-CA IX antibody is to work through ADCC and CDC, then it is preferable that the anti-CA IX antibody remains on the surface of the target tumor cell until the antibody Fc region binds to effector cells. Methods for determining whether an antibody bound to a cognate cell surface antigen remains on a cell surface or is internalized are well known in the art.

### Other Inhibitory Agents

Having identified the importance of the functional carbonic anhydrase domain of CA IX in its role in tumorigenesis, any agent that specifically inhibits carbonic anhydrase activity of CA IX on CA IX+tumorigenic cells can be utilized in the methods of the present invention. Candidate agents that inhibit carbonic anhydrase activity of a CA IX protein of interest may be derived from almost any source of chemical libraries, naturally occurring compounds, or mixtures of compounds. Exemplary sources of candidate inhibitors, synthesis of libraries of peptides, peptoids, and small organic molecules are described below. Any agent that is an inhibitor or antagonist of carbonic anhydrase activity of CA IX, for example, human CA IX or biologically active variant thereof, can be used in the treatment methods of the present invention. The inhibitor of carbonic anhydrase activity can be a peptide antagonist, a peptoid antagonist, or a small organic molecule antagonist. It is expected that some inhibitors will act at one or more of the three zinc-binding histidine residues within the carbonic anhydrase domain of the CA IX polypeptide (for example, at residues 226, 228, and 251 of human CA IX shown in SEQ ID NO:2), which are obligatory for the catalytic activity of the CA domain (Sly and Hu (1995) Annu. Rev. Biochem. 64:375-401). However, the use and appropriateness of such inhibitors of CA IX carbonic anhydrase activity for the purposes of the invention are not limited to any theories of mechanism of action of the inhibitor. It is sufficient for purposes of the invention that an inhibitor inhibit the carbonic anhydrase activity of CA IX, for example, human CA IX or biologically active variant thereof as defined herein below.

Analogs of peptides as used herein include peptides having one or more peptide mimics, for example peptoids that possess protein-like activity. Included within the definition are, for example, peptides containing one or more analogs of an amino acid (including, for example, unnatural amino acids), peptides with substituted linkages, as well as other modifications known in the art, both naturally occurring and not naturally occurring.

The term “small molecule” includes any chemical or other moiety that can act to affect biological processes. Small molecules can include any number of therapeutic agents presently known and used, or can be small molecules synthesized in a library of such molecules for the purpose of screening for function. Small molecules are distinguished from polymers and macromolecules by size and lack of polymerization. Small molecules can include peptides, peptoids, and small organic molecules.

The candidate inhibitors of CA IX carbonic anhydrase activity and libraries of candidate inhibitors for screening by the assays disclosed herein can be derived from any of the various possible sources of candidate inhibitors, such as for example, libraries of peptides, peptoids, and small molecules. The inhibitor could be a polypeptide presented by phage display. In general an inhibitor of CA IX carbonic anhydrase activity can be any molecule that may be capable of inhibiting CA IX carbonic anhydrase activity, or carbonic anhydrase activity of a variant CA IX polypeptide. Some libraries for screening can be subdivided into library pools for assaying inhibition of CA IX carbonic anhydrase activity by the assays disclosed herein. Some of each pool is assayed and some is saved for reassay, or to further subdivide into subpools, should a positive be identified. Generation of some of the possible libraries suitable for assay by the methods of the invention is described herein below.

Libraries that are peptide and peptoid inhibitors of CA IX carbonic anhydrase activity are made as follows. A “library” of peptides may be synthesized and used following the methods disclosed in U.S. Pat. No. 5,010,175, (the '175 patent) and in International Publication No. WO 91/17823. In the method of the '175 patent, a suitable peptide synthesis support, for example, a resin, is coupled to a mixture of appropriately protected, activated amino acids. The method described in WO 91/17823 is similar but simplifies the process of determining which peptides are responsible for any observed alteration of gene expression in a responsive cell. The methods described in WO 91/17823 and U.S. Pat. No. 5,194,392 enable the preparation of such pools and subpools by automated techniques in parallel, such that all synthesis and resynthesis may be performed in a matter of days.

Further alternative agents include peptide analogs and derivatives that can act as inhibitors of gene expression, or as ligands or antagonists. Some general means contemplated for the production of peptides, analogs or derivatives are outlined in Weinstein, ed. (1983) Chemistry and Biochemistry of Amino Acids, Peptides, and Proteins—A Survey of Recent Developments (Marcell Dekker, Inc., New York), herein incorporated by reference. Moreover, substitution of D-amino acids for the normal L-stereoisomers can be carried out to increase the half-lives of the molecules.

Peptoids, polymers comprised of monomer units of at least some N-substituted moieties, can act as small molecule inhibitors herein and can be synthesized as described in International Publication No. WO 91/19735. Presently preferred amino acid substitutes are N-alkylated derivatives of glycine, which are easily synthesized and incorporated into polypeptide chains. However, any monomer units that allow for the sequence specific synthesis of pools of diverse molecules are appropriate for use in producing peptoid molecules. The benefits of these molecules for the purpose of the invention are that they occupy different conformational space than a peptide and are more resistant to the action of proteases because their amide linkages are N-substituted.

Peptoids are easily synthesized by standard chemical methods. The preferred method of synthesis is the “submonomer” technique described by Zuckermann et al (1992) J. Am. Chem. Soc. 114:10646-7. Synthesis by solid phase techniques of heterocyclic organic compounds in which N-substituted glycine monomer units form a backbone is described in copending application entitled “Synthesis of N-Substituted Oligomers” filed on Jun. 7, 1995 and is herein incorporated by reference in full. Combinatorial libraries of mixtures of such heterocyclic organic compounds can then be assayed for the ability to inhibit enzyme activity, specifically carbonic anhydrase activity of CA IX or biologically active variant thereof.

Synthesis by solid phase of other heterocyclic organic compounds in combinatorial libraries is also described in copending application U.S. Ser. No. 08/485,006 entitled “Combinatorial Libraries of Substrate-Bound Cyclic Organic Compounds” filed on Jun. 7, 1995, herein incorporated by reference in its entirety. Highly substituted cyclic structures can be synthesized on a solid support by combining the submonomer method with powerful solution phase chemistry. Cyclic compounds containing one, two, three or more fused rings are formed by the submonomer method by first synthesizing a linear backbone followed by subsequent intramolecular or intermolecular cyclization as described in the same application.

As with the antagonist anti-CA IX antibodies of the invention, the other CA IX inhibitory agents of the invention interact with CA IX or biologically active variant thereof resulting in inhibition of CA IX carbonic anhydrase activity by 5%, 10%, 15%, 20%, 25%, 30%, 35%, preferably 40%, 45%, 50%, 55%, 60%, more preferably 70%, 75%, 80%, 85%, and most preferably 90%, 95%, 99%, or 100%. Assays for detecting inhibition of carbonic anhydrase activity are known in the art, including those assays disclosed elsewhere herein.

### Screening Assays for Detection of Other Inhibitory Agents and Antibodies Capable of Inhibiting CA IX Carbonic Anhydrase Activity

The present invention thus provides methods for identifying other agents that can serve as therapeutic agents for the treatment of cancers that are characterized by expression of the CA IX protein. The methods comprise screening assays whereby the ability of a test agent to inhibit carbonic anhydrase activity of the CA IX protein of interest is assessed. Where a candidate test agent is identified as having the ability to inhibit carbonic anhydrase activity in an in vitro assay, the agent can be further tested for its ability to inhibit phenotype transformation of cell lines expressing the CA IX protein of interest using cell-based assays known in the art, including the soft agar assay described herein in the Experimental section below. Inhibitory agents identified using in vitro assays can also be further tested using in vivo assays.

Thus, the invention provides a method (also referred to herein as a “screening assay”) for identifying inhibitors, i.e., candidate or test compounds or agents (e.g., peptides, peptidomimetics, small organic molecules, or other drugs; and suitable inhibitory antibodies) that bind to the CA IX protein of interest for example, human CA IX or biologically active variant thereof, and which have an inhibitory effect on carbonic anhydrase activity of this protein.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), ribosomes (Hudson and Souriau (2001) Expert. Opin. Biol. Ther. 1(5):845-855), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), and yeast Hudson and Souriau (2001) Expert. Opin. Biol Ther. 1(5):845-855).

Determining the ability of the test compound to bind to the CA IX protein can be accomplished, for example, by coupling the test compound with a radioisotope or enzymatic label such that binding of the test compound to the CA IX protein, more particularly to the carbonic anhydrase (CA) domain of this protein, can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In another embodiment, an assay of the present invention is a cell-free assay comprising contacting a CA IX protein, or biologically active portion thereof comprising the CA domain, with a test compound and determining the ability of the test compound to bind to the CA IX protein or biologically active portion thereof, more particularly to the CA domain. Binding of the test compound to the CA IX protein can be determined either directly or indirectly as described above. In a preferred embodiment, the assay includes contacting the CA IX protein or biologically active portion thereof comprising the CA domain with a known compound that binds CA IX protein to form an assay mixture, contacting the assay mixture with a test compound, and determining the ability of the test compound to preferentially bind to CA IX protein or biologically active portion thereof as compared to the known compound.

In the above-mentioned assays, it may be desirable to immobilize a CA IX protein to facilitate separation of complexed from uncomplexed forms of the protein, as well as to accommodate automation of the assay. In one embodiment, a fusion protein can be provided that adds a domain that allows the CA IX protein to be bound to a matrix. For example, glutathione-S-transferase/CA IX fusion proteins can be adsorbed onto glutathione sepharose beads (Sigma Chemical, St. Louis, Mo.) or glutathione-derivatized microtitre plates, which are then combined with the test compound or the test compound and the nonadsorbed CA IX protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of CA IX binding or activity determined using standard techniques.

In yet another embodiment, an assay is a cell-free assay comprising contacting CA IX protein or biologically active portion thereof comprising the CA domain with a test compound and determining the ability of the test compound to inhibit the carbonic anhydrase activity of the CA IX protein or biologically active variant thereof. Determining the ability of the test compound to inhibit the carbonic anhydrase activity of a CA IX protein or biologically active portion thereof can be accomplished, for example, by determining the ability of the CA IX protein or portion thereof to bind to a CA IX substrate molecule and inhibit enzymatic activity, as described, for example, in the Experimental section below.

In the above-mentioned assays, it may be desirable to immobilize a CA IX protein to facilitate separation of complexed from uncomplexed forms of the protein, as well as to accommodate automation of the assay. In one embodiment, a fusion protein can be provided that adds a domain that allows the CA IX protein to be bound to a matrix. For example, glutathione-S-transferase/CA IX fusion proteins can be adsorbed onto glutathione sepharose beads (Sigma Chemical, St. Louis, Mo.) or glutathione-derivatized microtitre plates, which are then combined with the test compound or the test compound and the nonadsorbed CA IX protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of CA IX binding or activity determined using standard techniques.

Other techniques for immobilizing proteins on matrices can also be used in the screening assays of the invention. For example, CA IX protein can be immobilized utilizing conjugation of biotin and streptavidin. Biotinylated CA IX molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a CA IX protein but which do not interfere with the catalytic action of the CA IX protein on its substrate molecule can be derivatized to the wells of the plate, and unbound CA IX protein trapped in the wells by antibody conjugation. Methods for detecting such complexes, in addition to those described above for the GST-immobilized complexes, include immunodetection of complexes using antibodies reactive with the CA IX protein, as well as enzyme-linked assays that rely on detecting an enzymatic activity associated with the CA IX protein or biologically active portion thereof.

In another embodiment, inhibitors of CA IX carbonic anhydrase activity are identified in a method in which a cell is contacted with a candidate compound and the carbonic anhydrase activity of CA IX protein in the cell is determined relative to carbonic anhydrase activity of CA IX protein in a cell in the absence of the candidate compound. When carbonic anhydrase activity is less (statistically significantly less) in the presence of the candidate compound than in its absence, the candidate compound is identified as an inhibitor of CA IX protein carbonic anhydrase activity. The level of CA IX protein carbonic anhydrase activity in the cells can be determined by methods described elsewhere herein for detecting such enzymatic activity. The cells expressing CA IX protein can be derived from a CA IX+cancer cell line that is positive for expression of this tumor-associated antigen. Alternatively, any suitable host cell can be utilized for recombinant expression of the particular CA IX protein of interest, for example, human CA IX of SEQ ID NO:2 (encoded by SEQ ID NO:1), or biologically active variant thereof as noted elsewhere herein. The CA IX protein of interest can alternatively be expressed as part of a fusion protein as noted herein below. Methods for recombinant expression of CA IX protein or biologically active variants thereof are known in the art. See, for example, the host cell expression systems disclosed in U.S. Pat. No. 6,051,226, herein incorporated by reference in its entirety. Such cell-based assays provide a method for assaying an antibody for the ability to inhibit carbonic anhydrase activity of a CA IX protein of interest. In this manner, a candidate antibody of interest that is to be tested for its inhibitory activity can be combined with a cell expressing CA IX protein or biologically active variant thereof under conditions suitable for detecting carbonic anhydrase activity, and then the level of the carbonic anhydrase activity of CA IX protein in the cell is determined relative to carbonic anhydrase activity of CA IX protein in a cell in the absence of the candidate antibody. Inhibition of carbonic anhydrase activity by the antibody would be indicative that the antibody is an inhibitor of CA IX carbonic anhydrase activity. Such candidate antibodies that can be tested using this method include existing antibodies as well as candidate antibodies yet to be identified. By “existing” antibodies is intended those antibodies that have been previously identified, including, but not limited to, those antibodies already known in the art. The cell-based screening methods of the present invention provide a means for assaying such antibodies for their ability to inhibit CA IX carbonic anhydrase activity.

In yet another aspect of the invention, the CA IX proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with CA IX protein (“CA IX-binding proteins” or “CA IX-bp”). Then one can test for inhibition of CA IX carbonic anhydrase activity.

Thus, the invention includes generating cRNA and cDNA libraries for screening for inhibition of carbonic anhydrase activity of CA IX, can require expression of recombinant CA IX polypeptides, which can alternatively be expressed as a fusion protein as noted elsewhere herein, and can also involve transforming a cell with the gene for a CA IX polypeptide, or fusion protein comprising a CA IX polypeptide, for expression in a screening assay. However, it is not necessary to overexpress CA IX in all the cell-based assays, as CA IX is endogenously expressed in cancer cell lines (i.e., CA IX+neoplastic cells) derived from cancers that are characterized by expression of CA IX. Exemplary systems for generating polypeptides or libraries useful for the screening methods of the invention would include, for example, any standard or useful mammalian, bacterial, yeast, or insect expression system, many of which are described in U.S. Pat. No. 6,204,370 with regard to production of recombinant CA IX polypeptides. Thus any CA IX polypeptide or peptide useful in the invention can be made by these or other standard methods.

Other items not specifically exemplified, such as plasmids, can be constructed and purified using standard recombinant DNA techniques described in, for example, Sambrook et al. (1989), Molecular Cloning, A Laboratory Manual (2d ed., Cold Spring Harbor Laboratory Press, Plainview, N.Y.), and Ausubel et al. (1994) Current Protocols in Molecular Biology (Greene Publishing Associates and John Wiley & Sons, New York, N.Y.) under the current regulations described in United States Department of Health and Human Services (HHS), National Institute of Health (NLH) Guidelines for Recombinant DNA Research. These references include procedures for the following standard methods: cloning procedures with plasmids, transformation of host cells, cell culture, plasmid DNA purification, phenol extraction of DNA, ethanol precipitation of DNA, agarose gel electrophoresis, purification of DNA fragments from agarose gels, and restriction endonuclease and other DNA-modifying enzyme reactions.

### Methods of Treatment

The antagonist anti-CA IX antibodies, antigen-binding fragments thereof, and other agents that inhibit carbonic anhydrase activity of CA IX or biologically active variant thereof are collectively referred to herein as CA IX inhibitory agents. These CA IX inhibitory agents are useful in the treatment of cancers that are characterized by expression of the CA IX protein. Such cancers include, but are not limited to, carcinomas, such as mammary, bladder, ovarian, uterine, cervical, endometrial, squamous cell and adenosquamous carcinomas; and head and neck cancers; mesodermal tumors, such as neuroblastomas and retinoblastomas; sarcomas, such as osteosarcomas and Ewing's sarcoma; and melanomas. Of particular interest are head and neck cancers, gynecologic cancers including ovarian, cervical, vaginal, endometrial and vulval cancers as well as gynecologic precancerous conditions, such as metaplastic cervical tissues and condylomas; gastrointestinal cancer, such as, stomach, colon and esophageal cancers; urinary tract cancer, such as, bladder and kidney cancers; skin cancer; liver cancer, prostate cancer; lung cancer; and breast cancer.

Thus, the invention provides methods for treating a subject having a cancer that is characterized by expression of the tumor-associated antigen CA IX. By “subject” is intended mammals, e.g., humans, dogs, cattle, horses, and the like. Preferably the subject undergoing treatment with the methods of the invention is human.

Subjects having a cancer characterized by expression of the CA IX protein can be determined by standard assays known in the art. The presence of CA IX antigen can be detected and/or quantitated using a number of well-defined diagnostic assays. Those in the art can adapt any of the conventional immunoassay formats, for example radioimmunoassays, immunohistochemical staining, immunoelectron and scanning microscopy using immunogold, among other techniques, to detect and/or quantitate MN antigen. Many other formats for detection of CA IX antigen are available. Those can be Western blots, ELISAs (enzyme-linked immunosorbent assays), RIAs (radioimmunoassay), competitive EIA or dual antibody sandwich assays, among other assays all commonly used in the diagnostic industry. In such immunoassays, the interpretation of the results is based on the assumption that the antibody or antibody combination will not cross-react with other proteins and protein fragments present in the sample that is unrelated to CA IX. Exemplary immunoassays that are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876; herein incorporated by reference.

Exemplary monoclonal antibodies that can be used for detection of CA IX expression in a sample obtained from a subject that is suspected of having existing neoplastic (tumor) cells or newly formed neoplastic cells that express this tumor-associated antigen are the monoclonal antibodies designated M75 and G250. A complete discussion of the M75 antibody is disclosed in U.S. Pat. No. 6,051,226, herein incorporated by reference in its entirety. A hybridoma that produces this representative CA IX-specific antibody was deposited at the American Type Culture Collection [ATCC; 10801 University Blvd., Manassas, Va. 20110-2209 (USA)] under ATCC Number HB 11128. The G250 antibody is well known to those of skill in the art. See, for example, Oosterwijk et al. (1996) Proc. Natl. Acad. Sci. USA 37:461; Uemura et al. (1994) Int. J. Cancer 56:609-614; Oosterwijk et al. (1995) Semin. Oncol. 22:34-41; herein incorporated by reference in their entirety. These MN/CA IX-specific monoclonal antibodies can be used to identify the CA IX protein and can be used to readily identify CA IX antigen in Western blots, in radioimmunoassays, and immunohistochemically, for example, in tissue samples that are fresh, frozen, or formalin-, alcohol-, acetone- or otherwised fixed and/or paraffin-embedded and deparaffinized. Such samples include tissue specimens, body fluids, tissue extracts, and cell extracts obtained from the subject who is a candidate for treatment with the methods of the invention. Preferred tissue specimens to assay by immunohistochemical staining include cell smears, histological sections from biopsied tissues or organs, and imprint preparations among other tissue samples. Biopsied tissue samples can be, for example, those samples removed by aspiration, bite, brush, cone, chorionic villus, endoscopic, excisional, incisional, needle, percutaneous punch, and surface biopsies, among other biopsy techniques.

The treatment methods of the invention comprise administering to a subject in need thereof a therapeutically effective dose or amount of an antagonist anti-CA IX antibody, antigen-binding fragment thereof, or other agent that inhibits carbonic anhydrase activity of CA IX or biologically active variant thereof. When administered in accordance with the methods of the present invention, these CA IX inhibitory agents exhibit anti-tumor activity. “By “anti-tumor activity” is intended that the individual undergoing therapy with the CA IX inhibitory agents of the invention will exhibit a favorable response with respect to the neoplastic or preneoplastic condition for which the individual is being treated. Examples of favorable responses include, but are not limited to a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Thus, these agents that target CA IX carbonic anhydrase activity can be used to promote a positive therapeutic response with respect to a cancer that is characterized by expression of this protein. By “positive therapeutic response” is intended an improvement in the disease in association with the anti-tumor activity of these agents, and/or an improvement in the symptoms associated with the disease.

Factors influencing the respective amount of a particular agent to be administered to a subject to achieve inhibition of carbonic anhydrase activity of CA IX, thereby inhibiting proliferation of neoplastic or phenotype transformation of preneoplastic cells expressing this antigen (i.e., CA IX+neoplastic or preneoplastic cells) include, but are not limited to, the particular cancer or proliferative disorder or disorder undergoing therapy, the severity of the disease, the history of the disease, and the age, sex, height, weight, health, and physical condition of the individual undergoing therapy. The amount of CA IX inhibitory agent that will constitute an inhibitory amount will also vary depending on such parameters as the particular inhibitory agent and its potency, the half-life of the inhibitory agent in the body, the rate of progression of the cancer or proliferative disorder being treated, the responsiveness of the condition to the dose of treatment or pattern of administration, the formulation, the attending physician's assessment of the medical situation, and other considerations such as prior administration of other therapeutics, or co-administration of any therapeutic that will have an effect on the inhibitory activity of the inhibitory agent or that will have an effect on carbonic anhydrase activity of CA IX.

In all cases, routine experimentation in clinical trials can determine specific ranges for optimal therapeutic effect, for each therapeutic agent and each administrative protocol, and administration to specific subjects can also be adjusted to within effective and safe ranges depending on the subject's condition and responsiveness to initial administrations.

In some embodiments of the invention, the method comprises administration of multiple doses of antagonist anti-CA IX antibody or antigen-binding fragment thereof, or multiple doses of a small molecule inhibitory agent (e.g., peptide, peptoid, or other small molecule). The method may comprise administration of 1, 2, 3, 4, 5, 6, 7, 8, 9, 10, 15, 20, 25, 30, 35, 40, or more therapeutically effective doses of a pharmaceutical composition comprising an antagonist anti-CA IX antibody or antigen-binding fragment thereof or a pharmaceutical composition comprising a small molecule inhibitory agent identified herein. The frequency and duration of administration of multiple doses of the particular pharmaceutical composition comprising an inhibitor of CA IX carbonic anhydrase activity can be readily determined by one of skill in the art. Moreover, treatment of a subject with a therapeutically effective amount of such an inhibitory agent can include a single treatment or, preferably, can include a series of treatments.

It will also be appreciated that the effective dosage of antibody or antigen-binding fragment thereof or other CA IX inhibitory agent used for treatment may increase or decrease over the course of a particular treatment. Changes in dosage may result and become apparent from the results of diagnostic assays.

“Co-administration” as used herein means administration of an antagonist anti-CA IX antibody, an antigen-binding fragment thereof, or other agent that inhibits carbonic anhydrase activity of CA IX or biologically active variant thereof according to the treatment methods of the invention in combination with a second therapeutic agent. The second therapeutic agent can be any therapeutic agent useful for treatment of the subject's condition. Suitable second therapeutic agents include, but are not limited to, methotrexate, tamoxifen, nelandron, nilutamide, adriamycin, 5FU, interferons, chemokines such as secondary lymphoid-tissue chemokine (CCL21), interferons, and cytokines, including interleukin-2 (IL-2), IL-12, IL-13, and IL-15, and granulocyte-macrophage colony stimulating factor (GM-CSF), and the like. The CA IX inhibitory agents can also be administered in combination with radiation therapy.

For example, inhibition of neoplastic cell proliferation and/or tumor growth using a tumor-associated antigen for the CA IX+cancer undergoing treatment, or using a vaccine comprising such a tumor-associated antigen, as a second therapeutic agent used in conjunction with a therapeutic agent of the invention that inhibits carbonic anhydrase activity of CA IX or variant thereof is contemplated. Additionally, for example, a first therapeutic agent can be a small molecule inhibitor of CA IX carbonic anhydrase activity, and a second therapeutic agent can be an antisense or ribozyme molecule against CA IX that, when administered in a viral or nonviral vector, will facilitate a transcriptional inhibition of CA IX that will complement the inhibitory activity of the small molecule. Peptides that are useful for generating a CA IX+tumor-specific cytotoxic T-lymphocyte (CTL) response, and vaccines comprising these peptides, are known in the art. See, for example, International Publication Nos. WO 01/98363 and WO 01/60317, herein incorporated by reference. Also see U.S. Pat. No. 6,204,370, where antisense sequences specific for CA IX are disclosed. Co-administration may be simultaneous, for example, by administering a mixture of the therapeutic agents, or may be accomplished by administration of the agents separately, such as within a short time period. Co-administration also includes successive administration of an inhibitor of CA IX carbonic anhydrase activity and one or more of another therapeutic agent. The second therapeutic agent or agents may be administered before or after the inhibitor of CA IX carbonic anhydrase activity. The second therapeutic agent may also be an inhibitor of CA IX carbonic anhydrase activity, which has particular advantages when administered with the first inhibitor. Dosage treatment may be a single dose schedule or a multiple dose schedule.

Therapeutically effective doses of agents that inhibit carbonic anhydrase activity of CA IX on neoplastic or preneoplastic cells expressing this protein can be administered using any medically acceptable mode of administration that results in an effective amount of the inhibitory agent being presented to the cells that are expressing this protein without incurring unacceptable adverse side effects. Examples of routes of administration include parenteral, e.g., intravenous, infusion, intradermal, subcutaneous, intramuscular, oral (e.g., inhalation), transdermal (topical), transmucosal, and rectal administration. In one embodiment, therapeutically effective doses of these therapeutic agents are injected directly into the tumor (intratumoral) or into a peritumor site. By “peritumor site” is meant a site less than about 15 cm from an outer edge of the tumor. Administration may be to one or more sites. Thus, the therapeutically effective doses of these agents can be administered at multiple sites within a tumor and/or surrounding a tumor.

The antagonist anti-CA IX antibodies and other CA IX inhibitory agents of the invention can be incorporated into an appropriate pharmaceutical composition that includes a pharmaceutically acceptable carrier for the agent. The pharmaceutical carrier for the agents may be the same or different for each agent. Suitable carriers may be large, slowly metabolized macromolecules such as proteins, polysaccharides, polylactic acids, polyglycolic acids, polymeric amino acids, amino acid copolymers, and inactive viruses in particles. Such carriers are well known to those of ordinary skill in the art. Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Publishing Co., New Jersey, 1991). Pharmaceutically acceptable carriers in therapeutic compositions may contain liquids such as water, saline, glycerol, and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier. Liposomes are described in U.S. Pat. Nos. 5,422,120 and 4,762,915, International Publication Nos. WO 95/13796, WO 94/23697, and WO 91/144445, and EP 524,968, and in Starrier (1975) Biochemistry (W. H. Freeman, San Francisco, Calif.), pages 236-240; Szoka et al. (1980) Biochim. Biophys. Acta. 600:1-18; Bayer et al. (1979) Biochim Biophys Acta. 550:46-473; Rivnay et al. (1987) Methods Enzymol. 149:119-123; Wang and Huang (1987) Proc. Natl. Acad. Sci. USA 84:7851-7855; and Plant et al. (1989) Anal Biochem. 176:420-426.

The pharmaceutically acceptable carrier or diluent may be combined with other agents to provide a composition either as a liquid solution, or as a solid form (e.g., lyophilized), which can be resuspended in a solution prior to administration. As previously noted, the composition can be administered by parenteral or nonparenteral routes.

The CA IX inhibitory agents of the present invention can be prepared and/or identified using the naturally occurring, full-length CA IX protein of interest, for example, human CA IX (SEQ ID NO:2, encoded by SEQ ID NO:1). Alternatively, these inhibitory agents can be prepared and/or identified using a biologically active variant of the CA IX protein of interest, for example, a biologically active variant of human CA IX. For purposes of the present invention, a biologically active variant of a naturally occurring CA IX protein is referred to as a “CA IX polypeptide variant,” a “polypeptide variant of the CA IX protein,” or a “CA IX protein variant.” CA IX polypeptide variants refer to polypeptides or proteins derived from the native or naturally occurring CA IX protein, for example, human CA IX protein of SEQ ID NO:2. CA IX polypeptide variants can be naturally occurring or can be generated by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants include CA IX fragments comprising a functional carbonic anhydrase domain, allelic variants, muteins, homologous orthologues, analogues and fusions of native CA IX polypeptide sequences. By “analogues” is intended analogues of either a native CA IX protein or a fragment of a native CA IX protein that comprise a native CA IX sequence and structure having one or more amino acid substitutions, insertions, or deletions. Peptides having one or more peptoids (peptide mimics) are also encompassed by the term analogues (WO 91/04282).

Of particular interest are polypeptide variants of human CA IX of SEQ ID NO:2, which may be naturally occurring (e.g., allelic variants that occur at the CA IX locus) or recombinantly produced (for example, muteins). Polypeptide variants of human CA IX also encompass homologues or orthologues of this native protein.

Any CA IX protein or CA IX polypeptide variant that is biologically active as noted below can be used to prepare and/or identify the inhibitory agents of the present invention. For purposes of the present invention, the term “CA IX polypeptide” encompasses the native or naturally occurring CA IX protein, for example, human CA IX, and biologically active variants of the native or naturally occurring CA IX protein, including CA IX fragments as noted below, where the biologically active variants have the functional and structural characteristics defined herein below.

Fragments or so-called “truncated forms” of a native or naturally occurring CA IX protein or of a CA IX polypeptide variant can be used to prepare and/or identify the inhibitory agents of the present invention as long as the fragments have a functional carbonic anhydrase domain as noted below. Functional fragments or truncated forms of a CA IX protein, or of a CA IX polypeptide variant, are referred to as “CA IX fragments,” or “CA IX polypeptide fragments.” These fragments or truncated forms of a CA IX protein or of a CA IX polypeptide variant are generated by removing amino acid residues from the full-length CA IX amino acid sequence (i.e., the sequence for the native CA IX protein or the sequence for the CA IX polypeptide variant), for example, using recombinant DNA techniques well known in the art and described elsewhere herein, and include N-terminal and C-terminal deletions of the full-length CA IX sequence. Because CA IX fragments are directly or indirectly derived from a native or naturally occurring CA IX protein, they represent one type of CA IX polypeptide variant as noted herein above.

For purposes of screening candidate inhibitory agents, including candidate antagonist anti-CA IX antibodies, for their ability to inhibit CA IX carbonic anhydrase activity, the full-length CA IX polypeptide variants, and fragments or truncated forms of a full-length CA IX protein or of a full-length CA IX polypeptide variant, should retain a functional carbonic anhydrase domain that provides for a carbonic anhydrase activity that is at least 20%, 25%, 30%, 35%, 40%, 45%, preferably at least 50%, 55%, 60%, 65%, 70%, 75%, more preferably at least 80% of the carbonic anhydrase activity of the naturally occurring CA IX protein, for example, human CA IX shown in SEQ ID NO:2. Fragments of a CA IX protein or fragments of a CA IX polypeptide variant suitable for preparing the anti-CA IX antibodies of the invention will comprise at least one inhibitory epitope that can be recognized by an antagonist anti-CA IX antibody, and thus form an antibody-antigen complex in the presence of this antibody whereby carbonic anhydrase activity of the CA IX polypeptide fragment is inhibited. Suitable epitopes to be included in a CA IX polypeptide fragment include those inhibitory epitopes comprising contiguous and/or discontinuous residues of the CA IX polypeptide fragment, for example, inhibitory epitopes comprising contiguous and/or discontinuous residues within the carbonic anhydrase domain. The CA IX inhibitory agents of the invention identified using the methods of the invention are suitable for treating a subject having a cancer characterized by expression of CA IX, or expression of a naturally occurring allelic variant of CA IX.

Native or naturally occurring CA IX proteins and CA IX polypeptide variants, including CA IX fragments (derived from the native or naturally occurring CA IX protein or derived from a full-length CA IX polypeptide variant) that are useful in the methods of the invention may be modified further so long as they retain CA IX carbonic anhydrase activity as noted below. Further modifications include, but are not limited to, phosphorylation, substitution of non-natural amino acid analogues, and the like. For the purposes of the present invention, CA IX polypeptide variants have an amino acid sequence that shares at least 70%, generally at least 75%, 80%, 85%, preferably about 90% to 95% or more, and more preferably about 98% or more sequence identity to the native or naturally occurring CA IX protein, where sequence identity is determined as noted herein below. Thus, for example, where the CA IX polypeptide variant is a variant of human CA IX, the CA IX polypeptide variant has an amino acid sequence that shares at least 70%, generally at least 75%, 80%, 85%, preferably about 90% to 95% or more, and more preferably about 98% or more sequence identity to human CA IX of SEQ ID NO:2. Furthermore, suitable CA IX polypeptide variants for purposes of the present invention will have a functional carbonic anhydrase domain that shares at least 70%, generally at least 75%, 80%, 85%, preferably about 90% to 95% or more, and more preferably about 98% or more sequence identity to the carbonic anhydrase domain of human CA IX, i.e., residues 135-414 of SEQ ID NO:2. A variant of the CA IX protein useful in preparing the compositions of the invention may differ from the native or naturally occurring CA IX protein, for example human CA IX of SEQ ID NO:2, by as few as 1-15, as few as 1-10, such as 6-10, as few as 5, as few as 4, 3, 2, or even 1 amino acid residue, so long as the functional carbonic anhydrase domain of the variant retains at least 70% sequence identity with residues 135-414 of SEQ ID NO:2. As previously noted, the CA IX polypeptide variant will comprise a functional carbonic anhydrase domain that provides for a carbonic anhydrase activity that is at least 20%, 25%, 30%, 35%, 40%, 45%, preferably at least 50%, 55%, 60%, 65%, 70%, 75%, more preferably at least 80% of the carbonic anhydrase activity of the native CA IX protein.

Methods for calculating sequence identity and similarity are known in the art. See, for example, Computer Analysis of Sequence Data, Part 1, ed. Griffin and Griffin (Humana Press, New Jersey, 1994), von Heinje (1987) Sequence Analysis in Molecular Biology (Academic Press, New York); and Gribskov and Devereux, eds. (1991) Sequence Analysis Primer (M Stockton Press, New York). In general, to determine the percent identity of two amino acid sequences, the sequences are aligned for optimal comparison purposes. The percent identity between the two sequences is a function of the number of identical positions shared by the sequences (i.e., percent identity=number of identical positions/total number of positions (e.g., overlapping positions)×100). For example, by a CA IX variant polypeptide having an amino acid sequence at least 95% “identical” to a reference CA IX amino acid sequence is intended that the amino acid sequence of the CA IX variant polypeptide is identical to the reference CA IX polypeptide sequence except that the polypeptide sequence of the CA IX variant can include up to five amino acid alterations per each 100 amino acids of the reference CA IX amino acid sequence. These alterations of the CA IX reference sequence may occur at the amino or carboxy terminal positions of the reference CA IX polypeptide sequence or anywhere between those terminal positions, interspersed either individually among residues in the reference CA IX polypeptide sequence or in one or more contiguous groups within the reference CA IX sequence.

The determination of percent identity between two sequences can be accomplished using a mathematical algorithm. For the purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489.

The CA IX polypeptide variants useful in preparing the compositions of the invention may be obtained by amino acid substitutions, deletions, truncations, and insertions. Preferred CA IX polypeptides variants have one or more conservative amino acid substitutions of the polypeptide of SEQ BD NO:2. For example, conservative amino acid substitutions may be made at one or more amino acid residues. Preferably, substitutions are made at nonessential amino acid residues, and preferably involve 1-15 residues, 1-10 residues, including 1, 2, 3, 4, 5, 6, 7, 8, 9, or 10 residue substitutions.

A “nonessential” amino acid residue is a residue that can be altered from the wild-type sequence of a CA IX protein (e.g., the sequence of SEQ ID NO:2) without altering one of the biological activities, whereas an “essential” amino acid residue is required for a given biological activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See, for example, Bowie et al. (1990) Science 247:1306, herein incorporated by reference. Preferably, such substitutions would not be made for conserved cysteine residues, such as the amino terminal contiguous cysteine residues.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm, taught in Smith and Waterman (1981) Adv. Appl. Math. 2:482-489).

The CA IX polypeptide variants useful in preparing and/or screening for the compositions of the invention may be isolated as naturally occurring variants, isolated after mutagenesis or recombinant manipulation, or be synthetically produced. Naturally occurring allelic variants can be identified with the use of well-known molecular biology techniques, such as, for example, with polymerase chain reaction (PCR) and hybridization techniques. Methods for such manipulations are generally known in the art. In addition, variants of the CA IX proteins can be prepared by mutagenesis or recombinant manipulations. Methods for mutagenesis and nucleotide sequence alterations are well known in the art. See, for example, Kunkel (1985) Proc. Natl. Acad. Sci. USA 82:488-492; Kunkel et al. (1987) Methods Enzymol. 154:367-382; U.S. Pat. No. 4,873,192; Walker and Gaastra, eds. (1983) Techniques in Molecular Biology (MacMillan Publishing Company, New York), and the references cited therein. Obviously, the mutations that will be made in the DNA encoding the variant must not place the sequence out of reading frame and preferably will not create complementary regions that could produce secondary MRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference.

Thus, the CA IX polypeptides useful in preparing the antagonist anti-CA IX antibodies and identifying other inhibitory agents encompass both naturally occurring proteins as well as variations and modified forms thereof. Such variants will continue to possess the desired CA IX activity, i.e., carbonic anhydrase activity, so that specificity of these inhibitory agents can be tested against the variant polypeptide. For the purposes of the present invention, a “CA IX polypeptide variant,” including CA IX polypeptide fragments, will exhibit at least 20%, 25%, 30%, 35%, 40%, 45%, preferably at least 50% of the CA IX carbonic anhydrase activity of the native CA IX polypeptide. Thus, for example, a variant of the CA IX polypeptide of SEQ ID NO:2 will exhibit at least 20%, 25%, 30%, 35%, 40%, 45%, preferably at least 50% of the carbonic anhydrase activity of the corresponding native CA IX polypeptide of SEQ ID NO:2. More typically, variants exhibit at least 60% of the native CA IX carbonic anhydrase activity; even more typically, variants exhibit at least 80% of the native CA IX carbonic anhydrase activity.

The deletions, insertions, and substitutions of the CA IX polypeptide sequences useful in preparing the CA IX inhibitory compositions disclosed herein are not expected to produce radical changes in the characteristics of the particular CA IX protein. However, when it is difficult to predict the exact effect of the substitution, deletion, or insertion in advance of doing so, one skilled in the art will appreciate that the effect will be evaluated by routine screening assays. That is, the carbonic anhydrase activity can be evaluated by standard assays known to those skilled in the art. See, for example, the assays disclosed in the Experimental section below.

Specific amino acids within the CA domain that are involved in carbonic anhydrase activity of the CA IX polypeptide that are essential for carbonic anhydrase activity of CA IX can be identified by methods known in the art. Such methods include alanine-scanning mutagenesis, molecular evolution (Crameri et al. (1996) Nat. Biotechnol. 14(3):315-319; Crameri et al. (1998) Nature 15:288-291; Patten et al. (1997) Curr. Opin. Biotechnol. 8:724-733; Stemmer (1994) Proc. Natl. Acad. Sci. USA 91:10747-51; Stemmer (1994) Nature 370:389-391), or site-directed mutagenesis. See, Cunningham et al. (1989) Science 244:1081. Resulting mutants can be tested for biological activity. Sites critical for activity can be determined by structural analysis such as crystallization, photoaffinity labeling, or nuclear magnetic resonance. See, deVos et al. (1992) Science 255:306 and Smith et al. (1992) J. Mol. Biol. 224:899. For example, the three histidine residues residing at positions 226, 228, and 251 of SEQ ID NO:2 are known to be critical for this enzymatic activity in human CA IX.

The following examples are offered by way of illustration and not by way of limitation.

### EXPERIMENTAL

### Example 1

### Glu-Tagged CA IX Purification Procedure

The glu-tagged CA domain portion of the human CA IX protein used in the Examples below was expressed in a soluble form in Baculovirus-infected Tn5 cells. Media containing the secreted, glu-tagged CA IX protein (CA IX-glu) was harvested and stored at −80° C. Baculovirus media containing CA IX-glu was thawed (at room temperature) and made 1 mM PMSF. It was concentrated 6 to 7 fold on ice, on a 10 kD Minisette (Filtron) membrane.

An Anti Glu-tag Affi-Gel 10 (Biorad) affinity chromatography column was prepared in advance according to the manufacturer's instructions, using Protein G purified monoclonal antibodies. At 4° C., this column was equilibrated in the following equilibration buffer: PBS/10% Glycerol/1% Octyl Glucoside. The concentrated media was filtered on a 0.22 μM CN membrane (Nalgene). Complete, EDTA-free Protease Inhibitor Cocktail (Roche Diagnostics #1873580) supplemented with 5 μg/ml of Leupeptin, and 10 μg/ml of E-64, was added. This material was loaded on the Anti Glu-tag Affi-Gel 10 column.

This column was washed with 10 CV of the equilibration buffer. It was subsequently eluted with 10 CV of the following elution buffer: 0.1 mg/ml EYMPTD peptide, custom manufactured by ResGen (#K0121 008) in PBS/10% Glycerol/1% Octyl Glucoside. The eluate was dialyzed vs PBS/10% Glycerol/1% Octyl Glucoside on a YM-10 Millipore membrane to eliminate the elution peptide and to concentrate the protein. Finally, the concentrated eluate was sterile filtered on a 0.22 μM Millipore (Durapore) membrane.

### Example 2

### Glu-Tagged, Full-Length CA IX Purification Procedure

The full-length, glu-tagged human CA IX protein used in Examples below was expressed internally in Baculovirus-infected Tn5 cells. Cells were harvested and stored at −80° C. Cells were thawed (at room temperature) and diluted 1/3 with the following lysis buffer: PBS/10% Glycerol/0.5% Triton-X, pH 7.4, with Complete, EDTA-free Protease Inhibitor Cocktail (Roche Diagnostics #1873580) supplemented with 5 μg/ml of Leupeptin, and 10 μg/ml of E-64.

Cells were then lysed in a glass homogenizer on ice. Lysate was centrifuged at 15,000×g for 1 hour at 4° C. The centrifuged supernatant of the lysate was processed further as follows. The lysate, diluted 1/3 with the lysis buffer, was applied on a p-Aminomethyl-benzenesulfonamide Agarose gel matrix (Sigma# A-0796) equilibrated in the above lysis buffer at 4° C. (Lysis buffer is equilibration buffer #1.) The column was washed with 10 CV of equilibration buffer #1 and, subsequently eluted with 10 CV of the following elution buffer #1: PBS/10% Glycerol/0.1 mM Acetazolamide (Sigma A-9842). After concentration on a YM-30 Millipore membrane, the eluate was diafiltered into the following equilibration buffer #2: PBS/10% Glycerol/1% Octyl Glucoside, pH 7.4.

An Anti Glu-tag Affi-Gel 10 (Biorad) affinity chromatography column was prepared in advance according to the manufacturer's instructions, using Protein G purified monoclonal antibodies. At 4° C., this column was equilibrated in equilibration buffer #2. The diafiltered eluate was loaded on the Anti Glu-tag Affi-Gel 10 column. It was washed with 10 CV of equlibration buffer #2, and then eluted with 10 CV of the following elution buffer #2: 1 mg/ml EYMPTD peptide, custom manufactured by ResGen (#K0121 008) in PBS/10% Glycerol/1% Octyl Glucoside. The eluate was dialyzed vs PBS/10% Glycerol/1% Octyl Glucoside on a YM-30 Millipore membrane to eliminate the elution peptide and to concentrate the protein. Finally, the concentrated eluate was sterile filtered on a 0.22 μM Millipore (Durapore) membrane.

### Example 3

### Low-Throughput Assay for Carbonic Anhydrase Activity of CA IX

The following low-throughput assay can used to screen candidate test agents for their ability to inhibit carbonic anhydrase activity of CA IX. This assay can be conducted using the full-length CA IX protein, or a partial-length CA IX polypeptide that includes a functional carbonic anhydrase domain. The CA IX protein can be glu-tagged as well.

### Prepare 4×Buffer and Filter:

100 nM HEPES, pH7.5
400 mM phenol red
400mM NaSO₄
   Bubble CO₂ into H₂O (CO₂—H₂O).

### In a 96-Well UV Transparent Plate:

aliquot 50 μl 4×buffer/well;
add 25 μl/well CA standard (bovine carbonic anhydrase [Sigma] or CA IX diluted in PBS or glu-tagged CA IX diluted in PBS;
add 25 μl/well test sample diluted in media or media alone;
include wells with 50 μl 4×buffer+25 μl PBS+25 μl media as background.
Start reaction by adding 100 μl/well CO₂—H₂O.
Read at 562 nm in kinetic mode for 15.0 min with mixing.

### CA Assay (Mini)

### Prepare Phenol Buffer:

40 mM HEPES, pH 8.3 at 4° C.

400 mM phenol red (Tissue Culture Grade)
Reformulate mAbs in 40 mM HEPES final (need 25 μl sample/well).
Bubble CO₂ into H₂O on ice for a minimum of 30 min (“CO₂—H₂O”).
Resuspend Mafenide to 50 mg/ml in 40 mM HEPES=203 mM (must be freshly prepared).
   Dilute Samples with 40 mM HEPES:
   As a negative control, make one of the samples the monoclonal antibody M75 (should not inhibit)

As a positive control, make one of the samples 200 μM Mafenide (50 μM final)

(should inhibit)
Dilute CA-glu to 80 nM in phenol buffer (20 nM final).
Add equal volume of diluted CA-glu to samples (1:2 dilution).
Degas on ice for 20 min.
Incubate 96-well UV Star plate on ice.
Set-up reader for 560 nm for 5 minute kinetic read with 3 second shaking in between.
Aliquot 50 μl of sample into wells on ice.
Pour CO₂—H₂O into reservoir.
Click “Read” on plate reader controls and begin counting.
Before plate reader door closes, add CO₂—H₂O (50 μl/well).
Read plate.

### Assay Details:

Can test up to 12 wells per run

Need to include “without CA-glu” and “with CA-glu” controls with every run

Each sample should be tested in triplicate or quadruplet

### CA Assay (Macro/Midi)

### Prepare Buffer:

40 mM HEPES, pH 8.3 at 4° C.
Reformulate mAbs in 40 mM HEPES buffer.
Dilute CA-glu to 80 nM in HEPES buffer (20 nM final).
Resuspend Mafenide to 50 mg/ml in 40 mM HEPES=203 mM (must be freshly prepared).
   Dilute Samples with 40 mM HEPES:

Include as a sample the monoclonal antibody M75 (should not inhibit)

Include as a sample 200 μM Mafenide (50 μM final) (should inhibit)
Include a sample with no Ab or Mafenide (HEPES only) to determine spontaneous rate of acidification
Mix samples and CA-glu 1:1 on ice.
Include a control set with no CA-glu.
Degas on ice for 20 min.
Bubble CO₂ into H₂O on ice for a minimum of 30 min on ice.
Add equal volume CO₂ saturated water to CA-glu/mAb samples on ice.
Start monitoring pH immediately. Monitor pH every 10 sec., and chart pH vs. time.

### Expected Results:

The control set with no enzyme should not show a change in pH over the time of the assay.
Isotype control sets and the M75 set should show no change in acidification kinetics compared to the spontaneous acidification sample (water+enzyme only; no Abs or inhibitors).
The Mafenide sample should inhibit (slow down) the rate of acidification.

### Example 4

### High-Throughput Assay for Carbonic Anhydrase Activity of CA IX

The following high-throughput assay can be used to screen candidate test agents for their ability to inhibit carbonic anhydrase activity of CA IX. The glu-tagged CA IX (CA IX-glu) is purified as noted in the examples above. As with the low-throughput assay, the full-length CA IX protein or a partial-length CA IX polypeptide that includes a functional carbonic anhydrase can be used. The substrate used in the reaction is fluorescein diacetate (FDA), catalog # F-103 (available from Molecular Probes). The reference standard is Acetazolamide, catalog #A-9842 (available from Sigma Chemicals). The assay buffer is 50 mM MES, pH=6.5, 0.05% Tween-20, 0.1 mM EDTA.

### Protocol

The following assay is carried out in a 384-well black polypropylene plate.
1) 1.25 μl of test sample in 100% Dimethyl sulfoxide is added to each well.
2) Dilute CA IX-glu enzyme stock to 83.3 nM in assay buffer. Add 30 μl of dilute enzyme stock to each well. Mix. For background wells, add 30 μl of assay buffer—no enzyme.
3) Dilute FDA substrate stock to 62.5 μM in assay buffer. Add 20 μl of dilute substrate stock to each well. Mix.
4) Incubate 2 hr at room temperature.
5) Read fluorescence at Excitation=485nm, Emission=520nm.

### Example 5

### Soft Agar Assay for Detection of Inhibition of Carbonic Anhydrase Activity

The following protocol can be used to test the ability of a candidate inhibitory agent for its ability to inhibit carbonic anhydrase activity of CA IX.
1) Coat each well of a non-tissue culture 96-well plate with 1.2% poly(HEMA). When the coating is dry, sterilize the plate.
2) Seed cells in a 96-well plate at a density of 350 to 500 cells in 100 μl per well. If desired, prior to plating, the cells can be pre-incubated with an antibody to be tested for inhibitory activity.
3) Add 300 μl of melted 4% noble agar (60° C.) to 840 μl of media (37° C.) and mix.
4) Dispense 50 μl of mixture to each well, doing one column of wells at a time, and mix to create a homogeneous suspension of cells in agar.
5) Allow agar to solidify for 10 to 15 minutes at room temperature.
6) Overlay agar with 100 μl of media per well and incubate plate at 37° C. for 7 to 14 days (depending on how fast the cells grow). If desired, chemical compounds or antibodies to be tested for inhibitory activity can be included in the media.
7) After 7 to 14 days, add 20 μl of Alamar Blue to each well and gently shake the plate at room temperature for 10 to 15 minutes.
8) Return the plate to the incubator and monitor every hour early in the incubation period.
9) Alamar Blue is metabolized by the cells and reduced over time.
10) Reduced Alamar dye is read at 530 nm Excitation, 590 nm Emission and the fluorescence units are used as an indicator of cell growth.

### Example 6

### Carbonic Anhydrase Activity Is Required for the Ability of CA IX to Contribute Toward the Transformed Phenotype

The first objective of this study was to determine if expression of the proteoglycan-like (PG) domain of human CA IX (residues 38-134 of SEQ ID NO:2) alone or the carbonic anhydrase (CA) domain of human CA IX (residues 135-414) alone is sufficient to confer a transformed phenotype to NIH-3T3 cells. If the CA domain alone was identified as being sufficient, the second objective was to determine if enzymatic activity is required to confer a transformed phenotype to NIH-3T3 cells.

cDNA expression constructs encoding fill-length human CA IX (SEQ ID NO:2; encoded by SEQ ID NO:1), the PG domain of human CA IX (residues 38-134 of SEQ ID NO:2), the CA domain of human CA IX (residues 135-414 of SEQ ID NO:2), or the CA domain of CA IX bearing mutations abrogating carbonic anhydrase enzymatic activity were made and transfected into NIH-3T3 cells. The complete constructs were as follows:

Full-length CA IX construct (CA-full). This construct encoded the full-length human CA IX. The coding and translated polypeptide sequences are shown in SEQ ID NOS:1 and 2, respectively.

Proteoglycan-like (PG) domain construct (CA-PG). This construct encoded the signal peptide (residues 1-37 of SEQ ID NO:2), the PG domain (residues 38-131 of SEQ ID NO:2, which includes most of the PG domain of SEQ ID NO:2 and encompasses the entire functional region of the PG domain, i.e., residues 53-111 of SEQ ID NO:2), a tri-peptide serine linker (included for technical reasons), the transmembrane domain (residues 415-433 of SEQ ID NO:2), and the intracellular or intracytoplasmic domain (residues 434-459 of SEQ ID NO:2). The coding and translated polypeptide sequences are shown in SEQ ID NOS:3 and 4, respectively.

Carbonic anhydrase (CA) domain construct (CA-CAH). This construct encoded the signal peptide (residues 1-37 of SEQ ID NO:2), the CA domain (residues 135-414 of SEQ ID NO:2), the transmembrane domain (residues 415-433 of SEQ ID NO:2), and the intracellular or intracytoplasmic domain (residues 434-459 of SEQ ID NO:2). The coding and polypeptide sequences are shown in SEQ ID NOS:5 and 6, respectively.

Glu-tagged CA domain construct (CA-CAHglu). This construct encoded the signal peptide (residues 1-37 of SEQ ID NO:2), a short series of residues from the non-functional region of the PG domain (residues 3845 of SEQ ID NO:2), a single alanine linker (included for technical reasons), residue 47 of SEQ ID NO:2 (from the non-functional region of the PG domain), the glu tag (EYMPME), a short series of residues from the non-functional region of the PG domain (residues 123-134 of SEQ ID NO:2), the CA domain (residues 135414 of SEQ ID NO:2), the transmembrane domain (residues 415433 of SEQ ID NO:2), and the intracellular or intracytoplasmic domain (residues 434-459 of SEQ ID NO:2). The coding and polypeptide sequences are shown in SEQ ID NOS:7 and 8, respectively.

Glu-tagged mutant CA domain construct (CA-CAHglumut). This construct encoded the same amino acid sequence as the glu-tagged CA domain construct with the exception of having the three histidine residues obligatory for carbonic anhydrase activity (i.e., residues 226, 228, and 251 of SEQ ID NO:2) mutated to three glutamine residues. The coding and polypeptide sequences are shown in SEQ ID NOS:9 and 10, respectively.

These transfected cells, an untransfected negative control cell line, and an src-transfected positive control cell line were then tested for their ability to grow in soft agar using the soft agar assay described in Example 5. Results are shown in Table 1 below.

The results confirm that full-length CA IX confers upon NIH-3T3 cells the ability to grow in soft agar (transformed phenotype). These results further demonstrate that the carbonic anhydrase domain of CA IX alone is sufficient to confer this transformed phenotype (the ability to grow in soft agar), while the proteoglycan-like (PG) domain alone is not sufficient. Furthermore, mutants of the CA domain engineered to lack carbonic anhydrase enzymatic activity do not confer a transformed phenotype onto transfected NIH-3T3 cells, suggesting that in addition to expression of the CA domain, carbonic anhydrase activity is also necessary for conferring the transformed phenotype.

These results indicate that targeting carbonic anhydrase activity of CA IX is likely to be beneficial therapeutically. Thus, protein-based therapeutics such as antibodies which inhibit CA IX carbonic anhydrase activity, or small molecule based therapeutics that inhibit CAIX carbonic anhydrase activity, may be effective therapeutic agents for cancers expressing CA IX.

Table 1 shows results of the soft agar assay for the indicated cell lines. The units presented are fluorescence units. Results and SD are shown from four separate experiments.

| | Hours | | | | | | | | | | | |
|---|---|---|---|---|---|---|---|---|---|---|---|---|
| Cells | 0.1 | 0.5 | 1 | 2.5 | 3.25 | 3.75 | 6.5 | 23 | 46 | 50 | 93.5 | 118 |
| NIH3T3 | −797.2 | −24.3 | 134.5 | −15.4 | 22.2 | 56.6 | 698.8 | 2662.3 | 5877.8 | 6207.5 | 10857.2 | 11649 |
| SRD3T3 | −1819.2 | 179.3 | 4979.9 | 16506.2 | 20901.1 | 23792.8 | 41044 | 42671.7 | 39125.6 | 38729.2 | 35209.9 | 31278.1 |
| CA-CAHglu | −3351.3 | −233.0 | 195.6 | 926.7 | 1299.9 | 1560.0 | 6386.5 | 16959.7 | 31983.2 | 32362.5 | 41426.7 | 39313.4 |
| CA-CAHglumut | −3443.6 | −275.1 | −67.5 | 30.9 | 148.5 | 235.7 | 1761.8 | 6100.6 | 12666.8 | 13322.5 | 20701.7 | 21205.1 |

SD

| | | | | | | | | | | | | |
|---|---|---|---|---|---|---|---|---|---|---|---|---|
| NIH3T3 | 4046.3 | 332.7 | 246.5 | 153.0 | 145.6 | 155.6 | 415.3 | 952.6 | 1760.9 | 1836.8 | 2706.4 | 2746.4 |
| SRD3T3 | 3718.6 | 639.7 | 2203.9 | 4256.7 | 5012.5 | 5525.3 | | 2226.3 | 3000.5 | 3020.2 | 3215.8 | 2994.4 |
| CA-CAHglu | 4598.4 | 415.7 | 432.6 | 482.7 | 523.6 | 566.0 | 1707.8 | 3332.2 | 4058.7 | 4572.8 | 3048.9 | 2220.5 |
| CA-CAHglumut | 5477.7 | 487.5 | 391.6 | 276.7 | 264.3 | 274.9 | 838.0 | 1715.4 | 2844.0 | 2899.9 | 3659.3 | 3354.1 |

| | Hours | | | | | | | | | |
|---|---|---|---|---|---|---|---|---|---|---|
| Cells | 0.1 | 1 | 2 | 3 | 4 | 6.5 | 21 | 27 | 53 | 82 |
| NIH3T3 | 2438.8 | 2903.9 | 3872.5 | 4498.3 | 4861.6 | 7870.2 | 20099.9 | 23250.1 | 40770 | 54238.91 |
| SRD3T3 | 4800.5 | 16972.9 | 50163.25 | 85271.4 | 97382.7 | 99999.0 | 99999.0 | 99999.0 | 99999 | 99999 |
| CA-CAHglu | 2037.7 | 2758.5 | 5063.888 | 7971.7 | 10232.9 | 24947.2 | 94833.1 | 98444.3 | 99999 | 99999 |
| CA-CAHglumut | 2150.1 | 2605.4 | 3696.4 | 4451.5 | 4922.0 | 8336.9 | 23367.9 | 27178.9 | 48517.3 | 64697.5 |

SD

| | | | | | | | | | | |
|---|---|---|---|---|---|---|---|---|---|---|
| NIH3T3 | 847.4 | 698.7 | 513.7 | 357.1 | 291.9 | 405.5 | 2096.0 | 2534.4 | 4388.3 | 83.1 |
| SRD3T3 | 3013.1 | 12328.7 | 19295.6 | 14372.9 | 9063.2 | 0.0 | 0.0 | 0.0 | 4939.7 | 0.0 |
| CA-CAHglu | 233.1 | 371.5 | 810.9 | 1313.4 | 1691.9 | 4253.9 | 8609.7 | 5020.4 | 0.0 | 0.0 |
| CA-CAHglumut | 559.0 | 436.1 | 371.0 | 268.0 | 211.9 | 297.9 | 2102.7 | 2494.6 | 4493.5 | 5987.4 |

| | Hours | | | | | | | | | | | |
|---|---|---|---|---|---|---|---|---|---|---|---|---|
| Cells | 2 | 30 | 33 | 48 | 53 | 56 | 58 | 72 | 75 | 78 | 92 | 122 |
| NIH3T3 | 1527 | 19063 | 20584 | 29891 | 26272 | 31601 | 34789 | 42687 | 34572 | 44366 | 47209 | 47717 |
| SW620 | 92540 | 79677 | 77985 | 69877 | 73833 | 68242 | 64867 | 57266 | 65466 | 54610 | 48883 | 49592 |
| CA-full | 1899 | 28057 | 30269 | 43239 | 37808 | 45227 | 49664 | 55376 | 48065 | 54176 | 48883 | 49592 |
| CA-PG | 1553 | 9012 | 9818 | 14287 | 12534 | 15124 | 16771 | 20377 | 16569 | 21587 | 24639 | 24687 |
| CA-CAH | 2251 | 49526 | 53595 | 68492 | 63717 | 68033 | 64867 | 57266 | 65466 | 54610 | 48883 | 49592 |

SD

| | | | | | | | | | | | | |
|---|---|---|---|---|---|---|---|---|---|---|---|---|
| NIH3T3 | 351 | 3322 | 3554 | 5149 | 4440 | 5365 | 5909 | 7090 | 5600 | 6625 | 3025 | 3218 |
| SW620 | 6981 | 0 | 0 | 0 | 0 | 0 | 0 | 0 | 0 | 0 | 0 | 0 |
| CA-full | 190 | 4897 | 5163 | 7037 | 5981 | 7090 | 7118 | 2919 | 6898 | 1439 | 0 | 0 |
| CA-PG | 146 | 1388 | 1459 | 2112 | 1851 | 2241 | 2542 | 2966 | 2385 | 3088 | 3436 | 3425 |
| CA-CAH | 259 | 6503 | 6920 | 2077 | 6219 | 504 | 0 | 0 | 0 | 0 | 0 | 0 |

| | Hours | | | | | | | | |
|---|---|---|---|---|---|---|---|---|---|
| Cells | 1 | 4 | 19 | 24 | 27 | 29 | 43 | 46 | 49 |
| NIH3T3 | −30 | −143 | 3582 | 5487 | 5004 | 5270 | 8594 | 11021 | 9601 |
| SRD3T3 | 645 | 6881 | 56503 | 70503 | 68940 | 69887 | 75151 | 68531 | 72533 |
| CA-full | 151 | 665 | 10146 | 15187 | 13688 | 14237 | 21910 | 27950 | 24146 |
| CA-PG | 243 | −126 | 4191 | 6519 | 5901 | 6252 | 10098 | 12993 | 11138 |
| CA-CAH | 506 | 1267 | 14492 | 22129 | 20057 | 21107 | 32042 | 41151 | 35512 |

SD

| | | | | | | | | | |
|---|---|---|---|---|---|---|---|---|---|
| NIH3T3 | 141 | 302 | 1174 | 1733 | 1503 | 1565 | 2236 | 2980 | 2521 |
| SRD3T3 | 218 | 2538 | 17500 | 12885 | 15353 | 14104 | 315 | 0 | 0 |
| CA-full | 335 | 663 | 3996 | 5836 | 5266 | 5510 | 8005 | 10368 | 8816 |
| CA-PG | 460 | 355 | 1436 | 2078 | 1869 | 1927 | 2641 | 3475 | 2968 |
| CA-CAH | 439 | 734 | 5187 | 7739 | 6932 | 7273 | 10222 | 12983 | 11214 |

| | Hours | | | | | | | |
|---|---|---|---|---|---|---|---|---|
| Cells | 52 | 63 | 68 | 75 | 88 | 93 | 98 | 100 |
| NIH3T3 | 10207 | 12919 | 14510 | 15500 | 19215 | 20957 | 22747 | 23099 |
| SRD3T3 | 70873 | 66691 | 64038 | 62893 | 57768 | 54304 | 50730 | 50238 |
| CA-full | 25397 | 31176 | 34404 | 36334 | 42923 | 42866 | 43380 | 43463 |
| CA-PG | 11715 | 14674 | 16336 | 17564 | 21326 | 23583 | 25535 | 25989 |
| CA-CAH | 37325 | 44207 | 47401 | 48961 | 52816 | 52425 | 50337 | 50052 |
| SD | | | | | | | | |
| NIH3T3 | 2669 | 3202 | 3878 | 3537 | 4105 | 4442 | 4872 | 4850 |
| SRD3T3 | 0 | 0 | 0 | 0 | 0 | 0 | 0 | 0 |
| CA-full | 9384 | 11080 | 12687 | 12599 | 11817 | 10115 | 7913 | 7518 |
| CA-PG | 3104 | 3653 | 4137 | 4115 | 4947 | 5232 | 5661 | 5707 |
| CA-CAH | 11652 | 12195 | 11431 | 10069 | 6528 | 3699 | 991 | 506 |

### Example 7

### ELISA Test to Detect Antibodies Against the CA Domain of CA IX

The antigen used in the ELISA test was a glu-tagged recombinant protein representing the CA domain portion of human CA IX (“CA-glu” protein), isolated as described in Example 1. The CA-glu protein was suspended in phosphate-buffered saline (PBS) at a concentration of 1 μg/ml, and 100 μl/well of this solution was dispensed into 96-well, flat-bottomed plates (Immulon 4 HBX). After incubation of the plates overnight at 4° C., the protein solution was removed and the wells were washed with PBST (PBS+0.05% Tween-20). The wells received 200 μl/well of Blocking Buffer (3% Carnation non-fat dry milk in PBS+0.01% Tween-20), and the plates were incubated for 1-2 hours at room temperature. The wells were again washed with PBST, and each well then received 100 μl of a solution containing an antibody or hybridoma supernatant to be tested. In the case of the hybridoma supernatants, the solutions consisted of 5-50 μl of supernatant brought up to a final volume of 100 μl with Blocking Buffer. After incubation of the plates for 1 hour at room temperature, the wells were washed with PBST. Each well received 100 μl of a secondary antibody solution (horse radish peroxidase-conjugated rabbit anti-mouse IgG (gamma); Zymed #61-6020; diluted 1:4000 in Blocking Buffer), and the plates we reincubated for 0.5-1 hour at room temperature. After a final wash of the wells with PBST, 100 μl/well of ODP peroxidase substrate (Sigma #P-9187; prepared according to the manufacturer's instructions) was added, and the plates were incubated at room temperature for 5-30 min to allow color development. The peroxidase reaction was stopped by the addition of 50 μl/well of 4 M H₂SO₄, and the reaction in each well was quantified as the optical density at 490 nm minus the optical density at 540 nm.

### Example 8

### Fluorescence-Activated Cell Sorting (FACS) Assay to Detect Antibody Reactivity Against Cell-Surface CA IX

To determine whether anti-CA-glu antibodies could bind the CA domain when the domain was present as part of the native molecule on the surface of cells, fluorescence-activated cell sorting was performed. The cell type utilized in the assay was the HT29 cell line (ATCC #HTB-38), derived from a human colorectal adenocarcinoma. For the assay, the cells were detached from tissue culture plates by treatment for approximately 30 minutes with 3 mM EDTA. The cells were collected by centrifugation and then resuspended in cold incubation buffer (PBS containing 1% bovine serum albumin). Antibodies or hybridoma supernatants to be tested were mixed with 2-5×10⁵ cells in cold incubation buffer in a final volume of 100 μl. As a negative control, cells were mixed with 10 μg/ml of nonspecific mouse IgG2b; for a positive control, the cells were mixed with 10 μg/ml of the anti-CA IX monoclonal antibody M75 (Pastoreková et al. (1992) Virology 187:620-626). After incubation for 30 min at 4° C., the cells were collected by centrifugation, washed three times with cold incubation buffer, and mixed with a detecting antibody (goat anti-mouse IgG antibody conjugated with phycoerythrin; CALTAG M30004-4; 0.2 μg in 100 μl cold incubation buffer per cell sample). After incubation of the cells with the secondary antibody for 30 min at 4° C., the cells were again collected by centrifugation and washed three times with cold incubation buffer. Each cell sample was resuspended in 300-500 μl of incubation buffer containing 0.5 μl/sample of propidium iodide solution (Roche #1 348 639). The samples were then analyzed utilizing a FACScaliber FACS machine (Becton Dickinson).

### Example 9

### Generation of Hybridomas

Female BALB/c mice were first immunized with cells expressing full-length recombinant human CA IX. To produce the cells for the immunization, Tn5 insect cells were infected with a recombinant baculovirus encoding the human CA IX protein. Two days after the infection, the cells were collected by centrifugation and resuspended in PBS to a final concentration of 5×10⁷ cells/ml. The cell suspension was emulsified with an equal volume of Freund's Incomplete Adjuvant (Sigma, F-5506), and 0.2 ml of the emulsion was injected intraperitoneally into each mouse. The mice were then boosted every 2-3 weeks with an intraperitoneal injection of 30 μg/mouse of recombinant CA-glu protein. For the first boost, the CA-glu protein was emulsified with an equal volume of Freund's Complete Adjuvant (Sigma, F-5881) prior to injection. In all subsequent intraperitoneal boosts, the protein was emulsified with Freund's Incomplete Adjuvant prior to injection. Eight days following each boost, blood was collected from each animal, serum was generated from the blood samples, and the sera were tested for the level of antibodies recognizing the CA-glu protein (titer) using the ELISA described in Example 7.

Mice with the highest serum titers against CA-glu after the third boost were given a final boost of 10 μg of the CA-glu protein. For this boost, the protein was injected intravenously into the tail vein as a suspension in PBS, without any added adjuvant. Three days later, the mice were sacrificed and the spleens removed. Splenocytes were isolated and fused with Sp2/0 myeloma cells, and the fusion mixes were plated in 96-well plates under conditions selective for growth of hybridomas. Conditioned media from the wells were screened for the presence of anti-CA IX antibodies using the ELISA described in Example 7. Control wells in the ELISA received either nonspecific mouse IgG2b rather than hybridoma supernatant (negative control for assay background), or samples of hybridoma supernatants previously shown to contain anti-CA-glu antibodies (positive controls). Selected supernatants were also assayed utilizing the FACS protocol described in Example 8.

### Example 10

### Antigenic Region Analysis of Human CA IX

The full-length human CA IX protein and the carbonic anhydrase domain (i.e., residues 135-414 of SEQ ID NO:2) were subjected to antigenic region analysis using the Bioannotator program of Vector NTI (see, for example, Vector TI Advance (VNTI Suite 8.0) available from InforMax). The antigencity plots for both sequences predicted an antigenic region within the carbonic anhydrase domain, where the 28-residue region of antigenicity corresponded to residues 229 to 256 of SEQ ID NO:2 (data not shown).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of this invention.

What is claimed is:

1. A method for identifying an agent capable of inhibiting carbonic anhydrase activity of a CA IX polypeptide, said agent being selected from the group consisting of an antagonist anti-CA IX antibody, an antigen-binding fragment of said antagonist anti-CA IX antibody, a peptide, a peptoid, and a small organic molecule, said method comprising
a) combining an agent to be tested with a cell expressing said CA IX polypeptide under conditions suitable for detecting carbonic anhydrase activity, and
b) assessing the ability of said agent to inhibit said carbonic anhydrase activity, whereby inhibition of said carbonic anhydrase activity by said agent is indicative that said agent is an inhibitor.

2. The method of claim 1, wherein said agent is an antagonist anti-CA IX antibody or an antigen-binding fragment thereof.

3. The method of claim 2, wherein said anti-CA IX antibody or said antigen binding fragment thereof specifically reacts with an inhibitory epitope of said CA IX polypeptide to form an antibody-antigen complex, whereby the formation of said complex results in inhibition of carbonic anhydrase activity of said CA IX polypeptide.

4. The method of claim 3, wherein said inhibitory epitope is selected from the group consisting of an inhibitory epitope comprising contiguous amino acid residues of the carbonic anhydrase domain of said CA IX polypeptide, an inhibitory epitope comprising discontinuous amino acid residues of said carbonic anhydrase domain, and an inhibitory epitope comprising both contiguous and discontinuous amino acid residues of said carbonic anhydrase domain.

5. The method of claim 4, wherein said mammal is a human and wherein said CA IX polypeptide is human CA IX of SEQ ID NO:2, wherein said carbonic anhydrase domain is represented by amino acid residues 135 to 414 of SEQ ID NO:2.

6. The method of claim 5, wherein said inhibitory epitope comprises at least one amino acid residue selected from the group consisting of residues 226, 228, and 251 of SEQ ID NO:2.

7. The method of claim 5, wherein said inhibitory epitope comprises at least 5 contiguous amino acid residues selected from the group consisting of residues 220 through 260 of SEQ ID NO:2.

8. The method of claim 7, wherein said inhibitory epitope comprises at least one amino acid residue selected from the group consisting of residues 226,228, and 251 of SEQ ID NO:2.

9. The method of claim 3, wherein said anti-CA IX antibody is a human anti-CA IX monoclonal antibody or antigen-binding fragment thereof.

10. The method of claim 3, wherein said anti-CA IX antibody is a humanized anti-CA IX monoclonal antibody or antigen-binding fragment thereof.

11. The method of claim 3, wherein said anti-CA IX antibody or antigen-binding fragment thereof further comprises a cytotoxin, a therapeutic agent, or a radioactive metal ion attached thereto.

12. The method of claim 3, wherein said antigen-binding fragment is selected from the group consisting of a Fab fragment, an F(ab′)₂ fragment, an Fv fragment, and a single-chain Fv fragment.

13. A method for inhibiting proliferation of neoplastic cells in a mammal, where said neoplastic cells are characterized by expression of CA IX protein, said method comprising administering to said mammal a therapeutically effective dose of an agent that inhibits carbonic anhydrase activity of said CA IX protein, wherein said agent is identified by the method of claim 1.

14. The method of claim 13, wherein said agent that inhibits carbonic anhydrase activity of said CA IX protein is an antibody that is produced by a recombinant CHO cell or myeloma cell.

15. The method of claim 14, wherein said myeloma cell is an Sp2 or NS0 myeloma cell.

16. A method for identifying an agent that inhibits carbonic anhydrase activity of a CA IX polypeptide having a functional carbonic anhydrase domain, said method comprising:
a) combining an agent to be tested with a cell expressing said CA IX polypeptide under conditions suitable for detecting carbonic anhydrase activity; and
b) assessing the ability of said agent to inhibit said carbonic anhydrase activity, whereby inhibition of said carbonic anhydrase activity by said agent is indicative that said agent is an inhibitor.

17. The method of claim 16, wherein said CA IX polypeptide is selected from the group consisting of:
a) human CA IX having the amino acid sequence set forth in SEQ ID NO:2;
b) the polypeptide set forth as residues 135-414 of SEQ ID NO:2;
c) a polypeptide having carbonic anhydrase activity, said polypeptide comprising a fragment of human CA IX, said fragment comprising a functional carbonic anhydrase domain, wherein said carbonic anhydrase domain shares at least 70% sequence identity with residues 135-414 of SEQ ID NO:2; and
d) a polypeptide variant of human CA IX of SEQ ID NO:2, wherein said polypeptide variant shares at least 70% sequence identity with SEQ ID NO:2, said polypeptide variant having a functional carbonic anhydrase domain, wherein said carbonic anhydrase domain shares at least 70% sequence identity with residues 135-414 of SEQ ID NO:2.

18. The method of claim 17, wherein said fragment comprises a functional carbonic anhydrase domain that shares at least 80% sequence identity with residues 135-414 of SEQ ID NO:2.

19. The method of claim 18, wherein said fragment comprises a functional carbonic anhydrase domain that shares at least 90% sequence identity with residues 135-414 of SEQ ID NO:2.

20. The method of claim 19, wherein said fragment comprises a functional carbonic anhydrase domain that shares at least 95% sequence identity with residues 135-414 of SEQ ID NO:2.

21. The method of claim 17, wherein said polypeptide variant shares at least 80% sequence identity with SEQ ID NO:2 and wherein said carbonic anhydrase domain shares at least 80% sequence identity with residues 135-414 of SEQ ID NO:2.

22. The method of claim 21, wherein said polypeptide variant shares at least 90% sequence identity with SEQ ID NO:2 and wherein said carbonic anhydrase domain shares at least 90% sequence identity with residues 135-414 of SEQ ID NO:2.

23. The method of claim 22, wherein said polypeptide variant shares at least 95% sequence identity with SEQ ID NO:2 and wherein said carbonic anhydrase domain shares at least 95% sequence identity with residues 135-414 of SEQ ID NO:2.

24. The method of claim 16, wherein said cell is a host cell selected from the. group consisting of COS, Chinese hamster ovary, NIH-3T3, 293, and derivatives thereof.

25. The method of claim 16, wherein said cell is a cell line or a cell of a tissue that naturally expresses said CA IX.

26. The method of claim 16, wherein the ability of the agent to inhibit carbonic anhydrase activity is detected using a soft agar assay.

27. An inhibitor of carbonic anhydrase activity of a CA IX polypeptide that has been identified according to the method of claim 16, wherein said inhibitor is selected from the group consisting of a peptide, a peptoid, and a small organic molecule.

28. A method for inhibiting proliferation of neoplastic cells in a mammal, where said neoplastic cells are characterized by expression of CA IX protein, said method comprising administering to said mammal a therapeutically effective dose of an agent that inhibits carbonic anhydrase activity of said CA IX protein, wherein said agent is identified by the method of claim 16.

29. A composition comprising an agent that is capable of inhibiting carbonic anhydrase activity of a CA IX polypeptide, said agent being selected from the group consisting of an antagonist anti-CA IX antibody, an antigen-binding fragment of said antagonist anti-CA IX antibody, a peptide, a peptoid, and a small organic molecule, wherein said agent is identified by combining an agent to be tested with a cell expressing said CA IX polypeptide under conditions suitable for detecting said carbonic anhydrase activity; and assessing the ability of said agent to inhibit said carbonic anhydrase activity, whereby inhibition of said carbonic anhydrase activity by said agent is indicative that said agent is an inhibitor.

30. The composition of claim 29, wherein said composition is a pharmaceutical composition.

31. The composition of claim 30 further comprising a pharmaceutically acceptable carrier.

32. A method for inhibiting proliferation of neoplastic cells in a mammal, where said neoplastic cells are characterized by expression of CA IX protein, said method comprising administering to said mammal a therapeutically effective amount of the composition of claim 29.

33. A polypeptide consisting essentially of amino acid residues 135 to 414 of SEQ ID NO:2.

34. An improved method of treating a subject having a neoplasm that is characterized by expression of CA IX protein, said method comprising administering a therapeutically effective amount of a pharmaceutical agent to said subject, wherein the improvement is that the pharmaceutical agent comprises an agent that inhibits carbonic anhydrase activity of said CA IX protein, wherein said agent is identified by the method of claim 1.

35. The method of claim 34, wherein said agent is a monoclonal antibody.

36. A monoclonal antibody that inhibits carbonic anhydrase activity of human CA IX or biologically active variant thereof that comprises a functional carbonic anhydrase domain, said functional carbonic anhydrase domain comprising a region of residues that is homologous to residues 229 to 256 of SEQ ID NO:2, wherein said antibody specifically reacts with an inhibitory epitope of said human CA IX or an inhibitory epitope of said biologically active variant thereof to form an antibody-antigen complex, whereby the formation of said complex results in inhibition of said carbonic anhydrase activity, wherein said inhibitory epitope of said human CA IX comprises at least one of residues 229 to 256 of SEQ ID NO:2, and wherein said inhibitory epitope of said biologically active variant thereof comprises at least one residue of the region of residues that is homologous to residues 229 to 256 of SEQ ID NO:2.

37. The monoclonal antibody of claim 36, wherein said inhibitory epitope of said human CA IX comprises at least two of residues 229 to 256 of SEQ ID NO:2 but less than 28 of residues 229 to 256 of SEQ ID NO:2.

38. A pharmaceutical composition comprising the antibody of claim 36 and a pharmaceutically acceptable carrier.

39. A method for assaying an antibody for the ability to inhibit carbonic anhydrase activity of a CA IX polypeptide, said method comprising
a) combining an antibody to be tested with a cell expressing said CA IX polypeptide under conditions suitable for detecting carbonic anhydrase activity; and
b) assessing the ability of said antibody to inhibit said carbonic anhydrase activity, whereby inhibition of said carbonic anhydrase activity by said antibody is indicative that said antibody is an inhibitor.

40. The method of claim 39, wherein said antibody is an existing antibody.


SEQUENCE LISTING (10 sequences)

SEQ ID NO: 1 - DNA, 1380 bp, Homo sapiens
  CDS (1)...(1380)
atggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctag

SEQ ID NO: 2 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 3 - DNA, 540 bp, Artificial Sequence
  Coding sequence for human CA IX proteoglycan domain construct
atggctcccctgtgccccagcccctggctccctctgttgatcccggcccttgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccataggagctcgagcatcctagccctggtttttggcctcctttttgctgtcaccagcgtcgcgttccttgtgcagatgagaaggcagcacagaaggggaaccaaagggggtgtgagctaccgcccagcagaggtagccgagactggagcctag

SEQ ID NO: 4 - protein, 179 aa, Artificial Sequence
  Polypeptide encoded by coding sequence for human CA IX proteoglycan domain construct
MAPLCPSPWLPLLIPALAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRSSSILALVFGLLFAVTSVAFLVQMRRQHRRGTKGGVSYRPAEVAETGA

SEQ ID NO: 5 - DNA, 1089 bp, Artificial Sequence
  Coding sequence for human CA IX carbonic anhydrase domain construct
atggctcccctgtgccccagcccctggctccctctgttgatcccggcccctgctccaggcctcactgtgcaactgctgctgtcactgctgcttctgatgcctgtccatcccggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctag

SEQ ID NO: 6 - protein, 362 aa, Artificial Sequence
  Polypeptide encoded by coding sequence for human CA IX carbonic anhydrase domain construct
MAPLCPSPWLPLLIPAPAPGLTVQLLLSLLLLMPVHPGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 7 - DNA, 978 bp, Artificial Sequence
  Coding sequence for glu-tagged human CA IX carbonic anhydrase domain construct
atggctcccctgtgccccagcccctggctccctctgttgatcccggcccctgctccaggcctcactgtgcaactgctgctgtcactgctgcttctgatgcctgtccatccccagaggttgccccggatgcaggaggctagcgaatacatgccaatggaacaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctag

SEQ ID NO: 8 - protein, 325 aa, Artificial Sequence
  Polypeptide encoded by coding sequence for glu-tagged human CA IX carbonic anhydrase domain construct
MAPLCPSPWLPLLIPAPAPGLTVQLLLSLLLLMPVHPQRLPRMQEASEYMPMEQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYELHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 9 - DNA, 978 bp, Artificial Sequence
  Coding sequence for glu-tagged mutant human CA IX carbonic anhydrase domain construct
atggctcccctgtgccccagcccctggctccctctgttgatcccggcccctgctccaggcctcactgtgcaactgctgctgtcactgctgcttctgatgcctgtccatccccagaggttgccccggatgcaggaggctagcgaatacatgccaatggaacaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcaactgcagtggggggctgcaggtcgtccgggctcggagcacactgtggaaggccaccgtttccctgccgagatccaagtggttcacctcagcaccgcctttgccagagttgacgaggccttggggcgcccgggaggcctggccgtgttggccgcctttctggaggagggcccggaagaaaacagtgcctatgagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctag

SEQ ID NO: 10 - protein, 325 aa, Artificial Sequence
  Polypeptide encoded by coding sequence for glu-tagged mutant human CA IX carbonic anhydrase domain construct
MAPLCPSPWLPLLIPAPAPGLTVQLLLSLLLLMPVHPQRLPRMQEASEYMPMEQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLQLQWGAAGRPGSEHTVEGHRFPAEIQVVHLSTAFARVDEALGRPGGLAVLAAFLEEGPEENSAYELHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA